(19) United States
(12) Patent Application Publication
(10) Pub. No.: US 2010/0234378 A1
(43) Pub. Date: Sep. 16, 2010

(54) ALKYLSULFONAMIDE-SUBSTITUTED TRIAZOLES AS MATRIX METALLOPROTEASE INHIBITORS

(75) Inventors: Andrew M. Fryer, Austin, TX (US); James Rizzi, Boulder, CO (US)

Correspondence Address: VIKSNINS HARRIS & PADYS PLLP, P.O. BOX 111098, ST. PAUL, MN, 55111-1098, US

(73) Assignee: ARRAY BIOPHARMA INC., Boulder, CO (US)

(21) Appl. No.: 12/438,339

(22) PCT Filed: Aug. 21, 2007
(86) PCT No.: PCT/US07/76434

§ 371 Date: May 21, 2009

Related U.S. Application Data
(60) Provisional application No. 60/839,337, filed on Aug. 22, 2006.

(51) Int. Cl. A61K 31/4192; C07D 249/04; C07D 403/12; C07D 413/12; A61K 31/5377; A61K 31/4439

(52) U.S. Cl. 514/236.2; 548/255; 544/132; 546/268.4; 514/359; 514/406; 514/340

(57) ABSTRACT

Provided are MMP-2, MMP-3, MMP-9, MMP-12 and/or MMP-13 inhibitors having the Formula (I): wherein R², R³, R⁴, R⁵, and Y are as defined herein, which are useful in the treatment and/or prevention of MMP mediated diseases and disorders.

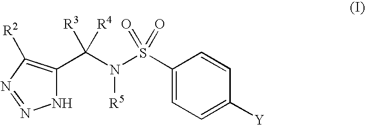

### PRIORITY OF INVENTION

This application claims priority to U.S. Provisional Application No. 60/839,337 that was filed on 22 Aug. 2006.

### BACKGROUND OF INVENTION

1. Field of Invention

The present invention relates to novel compounds, to pharmaceutical compositions comprising the compounds, to a process for making the compounds and to the use of the compounds in therapy. More particularly it relates to certain alkylsulfonamide-substituted triazoles that inhibit metalloproteases such as matrix metalloproteases (MMPs), which compounds are useful in the treatment of mammals having disease states alleviated by the inhibition of such metalloproteases such as, but not limited to, MMP-induced excessive degradation of matrix and connective tissue within the mammal, such as arthritis (rheumatoid arthritis and osteoarthritis), bone resorptive diseases (such as osteoporosis), and destruction of articular cartilage.

2. Description of the State of the Art

Matrix metalloproteases (“MMPs”) are a family of naturally-occurring proteases (enzymes) found in most mammals. MMPs are involved in the degradation and remodeling of connective tissues, and are present in various cell types that reside in or are associated with connective tissue, such as fibroblasts, monocytes, macrophages, endothelial cells, and invasive or metastatic tumor cells.

The MMPs share a number of properties, including zinc and calcium dependence. The MMPs are classified into several families based on their domain structure: matrilysin (minimal domain, MMP-7), collagenase (hemopexin domain, MMP-1, MMP-8, MMP-13), gelatinase (fibronectin domain, MMP-2, MMP-9), stromelysin (hemopexin domain, MMP-3, MMP-10, MMP-11), metalloelastase (MMP-12). In addition, the transmembrane domain family (MT-MMPs) has been recently discovered and includes MMP-14 through MMP-17.

The catalytic zinc domain in MMPs is typically the focal point for inhibitor design. Modification of substrates by introducing a zinc binding group (ZBG) has generated MMP inhibitors. Zinc binding groups in known MMP inhibitors include carboxylic acid, hydroxamic acid, sulfhydryl and mercapto groups.

Inhibition of matrix metalloproteases may be useful in the treatment of inflammatory diseases such as, arthritis (rheumatoid arthritis and osteoarthritis), bone resorptive diseases, such as osteoporosis, diseases characterized by abnormal blood vessel growth and remodeling, such as macular degeneration, diabetic retinopathy and restenosis, hyperproliferative diseases such as cancer, periodontitis, multiple sclerosis, chronic obstructive pulmonary disease, cerebral hemorrhaging associated with stroke, periodontal disease, aberrant angiogenesis, tumor invasion and metastasis, corneal and gastric ulceration, ulceration of skin, and aneurysmal disease. Inhibition of the activity of one or more MMPs may be of benefit in these diseases or conditions.

The need remains to find new low molecular weight compounds that are potent and selective MMP inhibitors, and that have an acceptable therapeutic index of toxicity/potency to make them amenable for use clinically in the prevention and treatment of the associated disease states.

### SUMMARY OF THE INVENTION

It has now been found that certain alkylsulfonamide-substituted triazoles compounds are inhibitors of the matrix metalloprotease MMP-13. Certain compounds are also inhibitors of other matrix metalloproteases, including but not limited to MMP-2, MMP-3, MMP-9 and/or MMP-12.

More specifically, one aspect of the invention provides compounds of Formula

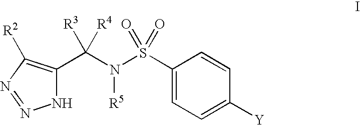

and enantiomers and salts thereof, wherein R², R³, R⁴, R⁵ and Y are as defined herein.

The compounds of the present invention can be used as prophylactics or therapeutic agents for treating diseases or disorders including, but not limited to, diseases and disorders mediated by MMP-2, MMP-3, MMP-9, MMP-12 or MMP-13. The compounds and compositions containing them are therefore useful in the treatment of diseases associated with the MMP-2, MMP-3, MMP-9, MMP-12 or MMP-13-induced excessive degradation of matrix and connective tissue within the mammal, such as arthritis (rheumatoid arthritis and osteoarthritis), bone resorptive diseases (such as osteoporosis), diseases characterized by abnormal blood vessel growth and remodeling such as macular degeneration, diabetic retinopathy and restenosis, hyperproliferative diseases such as cancer, periodontitis, multiple sclerosis, chronic obstructive pulmonary disease, cerebral hemorrhaging associated with stroke, periodontal disease, aberrant angiogenesis, tumor invasion and metastasis, corneal and gastric ulceration, ulceration of skin, and aneurysmal disease.

Another aspect of the present invention provides methods of preventing or treating a disease or disorder modulated by MMPs, comprising administering to a mammal in need of such treatment an effective amount of a compound of this invention or a stereoisomer or pharmaceutically acceptable salt thereof. Examples of such diseases and disorders include, but are not limited to, arthritis (rheumatoid arthritis and osteoarthritis), bone resorptive diseases (such as osteoporosis), and destruction of articular cartilage.

Another aspect of the present invention provides the use of a compound of this invention in the manufacture of a medicament for the treatment of a MMP modulated disease.

An additional aspect of the invention is the use of a compound of Formula I in the preparation of a medicament for treating MMP-2, MMP-3, MMP-9, MMP-12 or MMP-13-mediated diseases and conditions.

Another aspect of the present invention provides a pharmaceutical composition comprising a compound of this invention or a pharmaceutically acceptable salt thereof, and a pharmaceutically acceptable carrier or excipient.

Another aspect of the present invention includes methods of preparing, methods of separation, and methods of purification of the compounds of this invention.

### DETAILED DESCRIPTION OF THE INVENTION

Reference will now be made in detail to certain embodiments of the invention, examples of which are illustrated in the accompanying structures and formulas. While the invention will be described in conjunction with the enumerated embodiments, it will be understood that they are not intended to limit the invention to those embodiments. On the contrary, the invention is intended to cover all alternatives, modifications, and equivalents which may be included within the scope of the present invention as defined by the claims. One skilled in the art will recognize many methods and materials similar or equivalent to those described herein, which could be used in the practice of the present invention. The present invention is in no way limited to the methods and materials described. In the event that one or more of the incorporated literature and similar materials differs from or contradicts this application, including but not limited to defined terms, term usage, described techniques, or the like, this application controls.

### DEFINITIONS

The term “alkyl” as used herein refers to a saturated linear or branched-chain monovalent hydrocarbon radical of one to six carbon atoms, wherein the alkyl radical may be optionally substituted independently with one or more substituents described below. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (1-Bu, i-butyl, —CH₂CH(CH₃)₂)_{:} 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and the like.

The terms “cycloalkyl,” “carbocycle,” “carbocyclyl” and “carbocyclic ring” are used interchangeably and refer to a saturated or partially unsaturated cyclic hydrocarbon radical having from three to six carbon atoms. Examples of cycloalkyl groups include, cyclopropyl, cyclobutyl, cyclopentyl, cyclopentenyl, and cyclohexyl

The term “aryl” as used herein means a monovalent aromatic hydrocarbon radical of 6-10 carbon atoms derived by the removal of one hydrogen atom from a single carbon atom of a parent aromatic ring system. Aryl includes bicyclic radicals comprising an aryl radical fused to a saturated, partially unsaturated ring, or aromatic carbocyclic or heterocyclic ring. Exemplary aryl groups include, but are not limited to, radicals derived from benzene, naphthalene, anthracene, biphenyl, indene, indane, 1,2-dihydronapthalene, 1,2,3,4-tetrahydronapthalene, and the like. Aryl groups may be optionally substituted independently with one or more substituents described herein.

The term “heterocyclyl” refers to a saturated or partially unsaturated carbocyclic radical of 3 to 6 ring atoms in which at least one ring atom is a heteroatom independently selected from nitrogen, oxygen and sulfur, the remaining ring atoms being C, where one or more ring atoms may be optionally substituted independently with one or more substituents described below. The radical may be a carbon radical or heteroatom radical. Examples of heterocyclic rings include, but are not limited to, pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, homopiperazinyl, azetidinyl, oxetanyl, thietanyl, homopiperidinyl, oxepanyl, thiepanyl, oxazepinyl, diazepinyl, thiazepinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, and dihydrofuranyl.

The term “heteroaryl” as used herein refers to a monovalent aromatic radical of a 5-, 6-, or 7-membered ring, and includes fused ring systems (at least one of which is aromatic) of 5-12 atoms, containing at least one heteroatom independently selected from nitrogen, oxygen, and sulfur. Examples of heteroaryl groups include, but are not limited to, pyridinyl, imidazolyl, imidazopyridinyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Spiro moieties are also included within the scope of this definition.

The term “a” as used herein means one or more.

The terms “treat” or “treatment” refer to both therapeutic treatment and prophylactic or preventative measures, wherein the object is to prevent or slow down (lessen) an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

“Destruction of articular cartilage” as used herein refers to connective tissue disorders resulting in articular cartilage destruction, such as but not limited to, joint injury, reactive arthritis, acute pyrophosphate arthritis (pseudogout), psoriatic arthritis, juvenile rheumatoid arthritis, and osteoarthritis.

The phrases “therapeutically effective amount” or “effective amount” mean an amount of a compound of the present invention that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. The amount of a compound of Formula I that will correspond to such an amount will vary depending upon factors such as the particular compound, disease condition and its severity, the identity (e.g., weight) of the mammal in need of treatment, but can nevertheless be routinely determined by one skilled in the art.

The phrase “pharmaceutically acceptable” indicates that the substance or composition must be compatible chemically and/or toxicologically, with the other ingredients comprising a formulation, and/or the mammal being treated therewith.

The phrase “pharmaceutically acceptable salt,” as used herein, refers to pharmaceutically acceptable organic or inorganic salts of a compound of the invention.

The compounds of this invention also include other salts of such compounds which are not necessarily pharmaceutically acceptable salts, and which may be useful as intermediates for preparing and/or purifying compounds of this invention and/or for separating enantiomers of compounds of this invention.

The term “mammal” means a warm-blooded animal that has or is at risk of developing a disease described herein and includes, but is not limited to, guinea pigs, dogs, cats, rats, mice, hamsters, and primates, including humans.

### MMP Inhibitor Compounds

The present invention provides compounds, and pharmaceutical formulations thereof, that are potentially useful in the treatment of diseases, conditions and/or disorders modulated by MMPs, and more specifically MMP-2, MMP-3, MMP-9, MMP-12 or MMP-13.

One embodiment of the present invention provides compounds of Formula I:

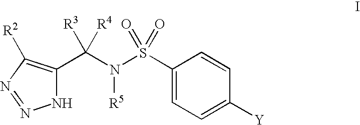

and enantiomers and salts thereof, wherein:

R² is H, Br, C₁, C₁-C₆ alkyl, Ar¹, or CH₂—Ar²;

R³ and R⁴ are independently H, C₁-C₆ alkyl, Ar³, CH₂—Ar⁴, or a 5-6 membered heteroaryl ring;

R⁵ is H, C₁-C₆ alkyl, (C₂-C₄ alkyl)OMe, or (C₂-C₄ alkyl)heterocyclyl;

Y is selected from the structures:

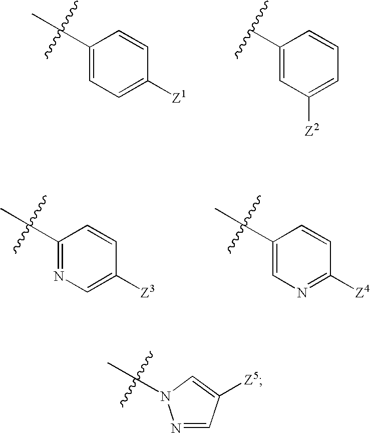

Z¹ is H, F, Cl, Br, CN, CF₃, C₁-C₆ alkyl, O—(C₁-C₆ alkyl), (C₁-C₆ alkyl)-OH,

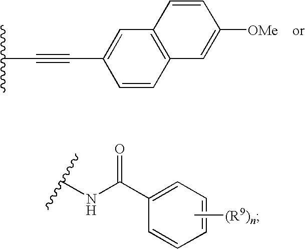

Z² is H, (C₁-C₃-alkyONH₂, CH₂NHR¹⁰, CH₂NHC(═O)OR⁷, or CH₂NHC(═O)R⁸;

Z³ is H, F, Cl, Br, CN, CF₃, C₁-C₆ alkyl, O—(C₁-C₆ alkyl), or (C₁-C₆ alkyl)-OH;

Z⁴ is H, CF₃, C₁-C₆ alkyl, or O—(C₁-C₆ alkyl);

Z⁵ is H, F, Cl, Br, CF₃, C₁-C₆ alkyl, or O—(C₁-C₆ alkyl);

R⁷ is C₁-C₆ alkyl, CH₂CH₂OMe, CH₂—Ar⁵, or a 3-6 membered cycloalkyl ring;

R⁸ is H, C₁-C₆ alkyl, CH₂-phenyl, a 3-6 membered cycloalkyl ring, C₆-C₁₀ aryl, or a 5-6-membered heteroaryl ring, wherein said aryl is optionally substituted with one to four R⁹ groups;

each R⁹ is independently F, Cl, CN, CF₃, C₁-C₆ alkyl, O—(C₁-C₆ alkyl), or (C₁-C₆ alkyl)-OH;

R¹⁰ is H or C₁-C₄ alkyl optionally substituted with a 5 or 6 membered aryl;

Ar¹, Ar², Ar³, Ar⁴ and Ar⁵ are each phenyl optionally and independently substituted with one or two groups independently selected from F, Cl, Br, I, C₁-C₃ alkyl, and O—(C₁-C₃ alkyl); and

n is 0, 1, 2, 3 or 4.

In a further embodiment, Z² is H, (C₁-C₃-alkyl)NH₂, CH₂NHC(═O)OR⁷, or CH₂NHC(═O)R⁸.

The compounds of this invention have been found to be inhibitors of MMP-13, and are useful in treating disease states alleviated by the inhibition of such metalloproteases, such as osteoarthritis.

In certain embodiments of Formula I, R⁵ is H, methyl, ethyl, CH₂CH₂OMe, or CH₂CH₂—(morpholin-4-yl). In particular embodiments, R⁵ is H.

In particular embodiments, R⁵ is methyl.

In particular embodiments, R⁵ is CH₂CH₂OMe.

In particular embodiments R⁵ is CH₂CH₂— (morpholin-4-yl).

In certain embodiments of Formula I, R² is H, Br, or phenyl. In particular embodiments, R² is H.

In particular embodiments, R² is Br.

In particular embodiments, R² is phenyl.

In certain embodiments of Formula I, R³ and R⁴ are independently H, phenyl, methyl, ethyl, isopropyl, benzyl, 2-pyridyl, 3-pyridyl or 4-pyridyl. In particular embodiments, R³ and R⁴ are H. In other embodiments, R³ and R⁴ are methyl. In other embodiments, R³ and R⁴ are ethyl. In other embodiments, R³ is H and R⁴ is methyl, ethyl, isopropyl or phenyl.

In certain embodiments of Formula I, Y is:

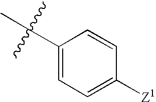

wherein Z¹ is as defined above. In certain embodiments, Z′ is H, F, Cl, CN,

CF₃, methyl, ethyl, isopropyl, OCH₃, OCH₂CH₃, OCH(CH₃)₂, CH₂OH, or

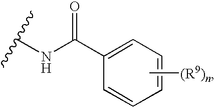

wherein each R⁹ is independently F, Cl, OCH₃, CH₂OH, CN or CF₃. Further exemplary embodiments of Z¹ include the following structures:

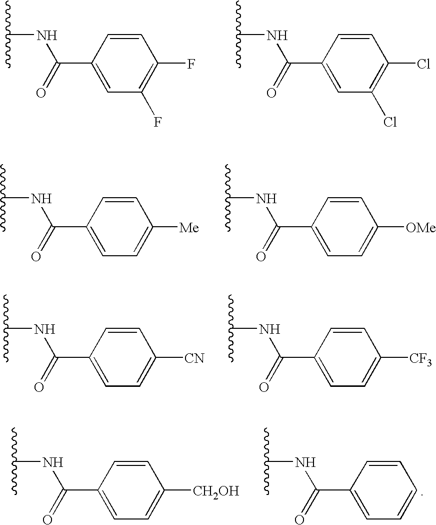

In other embodiments of Formula I, Y is:

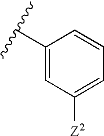

wherein Z² is as defined above. In certain embodiments, Z² is CH₂NH₂ or CH₂CH₂NH₂. In other embodiments, Z² is CH₂NHC(═O)OR⁷ wherein R⁷ is as defined above In particular embodiments, R⁷ is methyl, ethyl, isopropyl, t-butyl, cyclopentyl, CH₂CH₂OCH₃, or CH₂-phenyl. In other embodiments, Z² is CH₂NHC(═O)R⁸ wherein R⁸ is as define above. In particular embodiments, R⁸ is H, methyl, ethyl, isopropyl, t-butyl, cyclopropyl, cyclobutyl, cyclopentyl, cyclohexyl, 2-pyridyl, 3-pyridyl, 4-pyridyl, benzyl, naphthyl, phenyl, 4-fluorophenyl, 4-chlorophenyl, 4-methylphenyl, 4-methoxy phenyl, 4-(hydroxymethyl)phenyl, 4-cyanomethyl, or 4-(trifluoromethyl)phenyl.

In certain embodiments, Z² is CH₂NHR¹⁰. In certain embodiments, R¹⁰ is H or C₁-C₄ alkyl optionally substituted with a 5 or 6 membered aryl. In particular embodiments R¹⁰ is H or C₁-C₄ alkyl optionally substituted with phenyl. In particular embodiments, R¹⁰ is CH₂-phenyl. In particular embodiments, Z² is CH₂NHCH₂-phenyl.

In certain embodiments of Formula I, Y is:

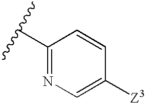

wherein Z³ is as defined above. In certain embodiments, Z³ is H, F, Cl, Br, methyl, ethyl, propyl, isopropyl, OCH₃, OCH₂CH₃, OCH(CH₃)₂, CH₂OH, CN or CF₃.

In certain embodiments of Formula I, Y is:

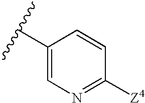

wherein Z⁴ is as defined above. In certain embodiments, Z⁴ is H, Me, Et, OCH₃, OCH₂CH₃, or CF₃.

In certain embodiments of Formula I, Y is:

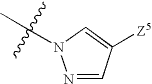

wherein Z⁵ as defined above. In particular embodiments, Z⁵ is H, F, Cl, Br, Me, Et, OMe, OEt, or CF₃.

It will be appreciated that certain compounds according to the invention may contain one or more centers of asymmetry and may therefore be prepared and isolated in a mixture of isomers such as a racemic mixture, or in an enantiomerically pure form, and accordingly that any such enantiomeric pure form is included within the scope of the present invention.

In the structures shown herein, where the stereochemistry of any particular chiral atom is not specified, then all stereoisomers are contemplated and included as the compounds of the invention. Where stereochemistry is specified by a solid wedge or dashed line representing a particular configuration, then that stereoisomer is so specified and defined.

It will further be appreciated that the compounds of Formula I or their salts may be isolated in the form of solvates, and accordingly that any such solvate is included within the scope of the present invention.

The compounds of Formula I include pharmaceutically acceptable salts thereof. The compounds of Formula I also include other salts of such compounds which are not necessarily pharmaceutically acceptable salts, and which may be useful as intermediates for preparing and/or purifying compounds of Formula I and/or for separating enantiomers of compounds of Formula I.

The present invention also embraces isotopically-labeled compounds of the present invention which are identical to those recited herein, but for the fact that one or more atoms are replaced by an atom having an atomic mass or mass number different from the atomic mass or mass number usually found in nature.

### Synthesis of Compounds

Compounds of the present invention may be synthesized by synthetic routes that include processes analogous to those well-known in the chemical arts, particularly in light of the description contained herein. The starting materials are generally available from commercial sources such as Sigma-Aldrich (St. Louis, Mo.), Alfa Aesar (Ward Hill, Mass.), or TCI (Portland, Oreg.), or are readily prepared using methods well known to those skilled in the art (e.g., prepared by methods generally described in Louis F. Fieser and Mary Fieser, Reagents for Organic Synthesis, v. 1-19, Wiley, N.Y. (1967-1999 ed.), or Beilsteins Handbuch der organischen Chemie, 4, Aufl. ed. Springer-Verlag, Berlin, including supplements (also available via the Beilstein online database)).

According to another aspect, the present invention provides a process for the preparation a compound of Formula I or a salt thereof as defined hereinabove, which comprises:

(a) coupling a compound having the Formula II:

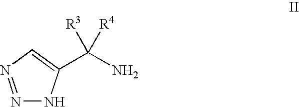

wherein R³ and R⁴ are as defined herein, with a compound having the Formula III:

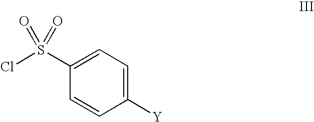

wherein Y is as defined herein, in the presence of a base; or

(b) reacting a compound having the Formula IV:

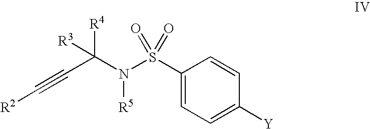

wherein R³, R⁴, R⁵, and Y are as defined herein and R² is as defined herein with the exception that R² is not Br, with azidotrimethylsilane in the presence of a catalytic amount of CuI; or

(c) for compounds of Formula I wherein R² is Br and at least one of R³ and R⁴ is H. reacting a compound of Formula I wherein R² is H with bromine; and

removing any protecting group or groups and, if desired, forming a salt.

Referring to the above method (a), a suitable base includes an organic base such as pyridine or triethylamine. In certain embodiments, the organic base also serves as the solvent for the reaction. The reaction can be conveniently performed at ambient temperature.

Compounds of Formula II are known or can be prepared by reacting a compound of the Formula V:

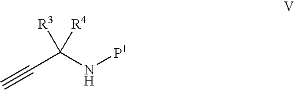

wherein R³ and R⁴ are as defined herein and P′ is H or an amine protecting group, for example Boc, with azidotrimethylsilane in the presence of a catalytic amount of CuI.

Referring to the above method (b), the reaction can be performed at an elevated temperature, such as in the range of from 70° C. to 100° C., for example between 85° C. to 95° C. Suitable solvents include sulfoxides, amides and alcohols, such as DMF, methanol, or mixtures thereof.

Compounds of the Formula IV, wherein R⁵ is H, can be prepared by reacting a compound of Formula VI:

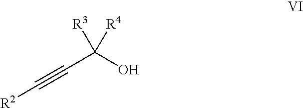

with a compound of the Formula VII:

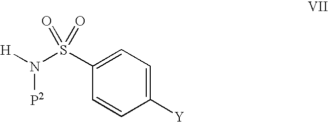

wherein P² is as defined for P¹, under standard coupling conditions, such as in the presence of diispropyl azodicarbocylate and triphenyl-phosphine. Compounds of Formula IV wherein R⁵ is as defined herein with the exception that R⁵ is not H can be prepared by reacting a compound of Formula IV with R⁵X, wherein X is a leaving group such as Cl, Br, I, OTf or OTs, in the presence of a base. The base may be, for example, an alkali metal carbonate, such as sodium carbonate or potassium carbonate.

In preparing compounds of Formula I, protection of remote functionalities (e.g., primary or secondary amines, etc.) of intermediates may be necessary. The need for such protection will vary depending on the nature of the remote functionality and the conditions of the preparation methods. Suitable amino-protecting groups (NH-Pg) include acetyl, trifluoroacetyl, t-butoxy carbonyl (BOC), benzyloxycarbonyl (CBz) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

### Methods of Separation

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., an enantiomer, substantially free of its stereoisomer may be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994; Lochmuller, C. H., (1975) J. Chromatogr., 113(3):283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts may be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem., (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111).

By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. of Chromatogr., (1990) 513:375-378). Enriched or purified enantiomers can be distinguished by methods used to distinguish other chiral molecules with asymmetric carbon atoms, such as optical rotation and circular dichroism.

### Administration and Pharmaceutical Formulations

The compounds of the invention may be administered by any convenient route, e.g. into the gastrointestinal tract (e.g. rectally or orally), the nose, lungs, musculature or vasculature or transdermally. The compounds may be administered in any convenient administrative form, e.g. tablets, powders, capsules, solutions, dispersions, suspensions, syrups, sprays, suppositories, gels, emulsions, patches, etc. Such compositions may contain components conventional in pharmaceutical preparations, e.g. diluents, carriers, pH modifiers, sweeteners, bulking agents, and further active agents. If parenteral administration is desired, the compositions will be sterile and in a solution or suspension form suitable for injection or infusion. Such compositions form a further aspect of the invention.

A typical formulation is prepared by mixing a compound of the present invention and a carrier or excipient. Suitable carriers and excipients are well known to those skilled in the art and are described in detail in, e.g., Howard C. Ansel et al., Pharmaceutical Dosage Forms and Drug Delivery Systems, (8^{th} Ed. 2004); Alfonso R. Gennaro et al., Remington: The Science and Practice of Pharmacy, (20^{th} Ed. 2000); and Raymond C. Rowe, Handbook of Pharmaceutical Excipients, (5^{th} Ed. 2005). The formulations may also include one or more buffers, stabilizing agents, surfactants, % vetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a compound of the present invention or pharmaceutical composition thereof) or aid in the manufacturing of the pharmaceutical product (i.e., medicament).

One embodiment of the present invention includes a pharmaceutical composition comprising a compound of Formula I, or a stereoisomer or pharmaceutically acceptable salt thereof. In a further embodiment, the present invention provides a pharmaceutical composition comprising a compound of Formula I, or a stereoisomer or pharmaceutically acceptable salt thereof, together with a pharmaceutically acceptable carrier or excipient.

According to another aspect, the present invention provides a compound of Formula I or a pharmaceutically acceptable salt thereof, for use in therapy.

According to a further aspect, the present invention provides the use of a compound of Formula I or a pharmaceutically acceptable salt thereof, in the manufacture of a medicament to treat an immunologic disorder, as defined hereinabove.

Methods of Treatment with Compounds of the Invention

The compounds of the present invention can be used as prophylactics or therapeutic agents for treating diseases or disorders including, but not limited to, diseases and disorders mediated by MMP-2, MMP-3, MMP-9, MMP-12 or MMP-13. The compounds and compositions containing them are therefore useful in the treatment of diseases associated with the MMP-2, MMP-3, MMP-9, MMP-12 or MMP-13-induced excessive degradation of matrix and connective tissue within the mammal, such as arthritis (rheumatoid arthritis and osteoarthritis), bone resorptive diseases (such as osteoporosis), destruction of articular cartilage, periodontitis, multiple sclerosis, chronic obstructive pulmonary disease, cerebral hemorrhaging associated with stroke, periodontal disease, aberrant angiogenesis, tumor invasion and metastasis, corneal and gastric ulceration, ulceration of skin, aneurysmal disease, and in complications of diabetes.

The compounds of the present invention can be used as prophylactics or therapeutic agents for treating diseases or disorders including, but not limited to, diseases and disorders mediated by MMP-13. The compounds and compositions containing them are therefore useful in the treatment of diseases associated with the MMP-13-induced excessive degradation of matrix and connective tissue within the mammal, such as arthritis (rheumatoid arthritis and osteoarthritis), bone resorptive diseases (such as osteoporosis), and destruction of articular cartilage.

Accordingly, another aspect of the invention provides methods of treating or preventing diseases or conditions described herein by administering to a mammal, such as a human, a therapeutically effective amount of a compound of Formula I.

In another embodiment of the present invention, the use of a compound of Formula I in the preparation of a medicament for treating MMP-2, MMP-3, MMP-9, MMP-12 or MMP-13-mediated diseases and conditions is provided.

An additional aspect of the invention is the use of a compound of Formula I in the preparation of a medicament for treating MMP-13-mediated diseases and conditions.

The ability of the compounds of Formula I to inhibit matrix metalloprotease activity may be demonstrated by a variety of in vitro assays known to those of ordinary skill in the art, such as the MMP Enzymatic Assay described in FEBS, 296, 263, (1992) or modifications thereof as described in more detail in Example A.

### Combination Therapy

The compounds of this invention and stereoisomers and pharmaceutically acceptable salts thereof may be employed alone or in combination with other therapeutic agents for treatment. The compounds of the present invention can be used in combination with one or more additional drugs, for example an anti-inflammatory compound that works by a different mechanism of action. The second compound of the pharmaceutical combination formulation or dosing regimen preferably has complementary activities to the compound of this invention such that they do not adversely affect each other. Such molecules are suitably present in combination in amounts that are effective for the purpose intended. The compounds may be administered together in a unitary pharmaceutical composition or separately and, when administered separately this may occur simultaneously or sequentially in any order. Such sequential administration may be close in time or remote in time.

The compounds of the present invention can be used in combination with one or more additional drugs for treating MMP-13-mediated conditions as defined above. The compounds of the present invention can also be used in combination with one or more additional drugs for treating MMP-2, MMP-3, MMP-9, MMP-12 or MMP-13-mediated conditions as defined above. Accordingly, another aspect of the present invention provides a composition comprising a compound of this invention in combination with a second drug, such as described herein.

### EXAMPLES

In order to illustrate the invention, the following examples are included. In the examples described below, unless otherwise indicated all temperatures are set forth in degrees Celsius. Reagents were purchased from commercial suppliers such as Aldrich Chemical. Company, Lancaster, TCI or Maybridge, and were used without further purification unless otherwise indicated.

The reactions set forth below were done generally under a positive pressure of nitrogen or argon or with a drying tube (unless otherwise stated) in anhydrous solvents, and the reaction flasks were typically fitted with rubber septa for the introduction of substrates and reagents via syringe. Glassware was oven dried and/or heat dried.

Column chromatography was done on a Biotage system (Manufacturer: Dyax Corporation) having a silica gel column or on a silica SepPak cartridge (Waters). ¹H NMR spectra were recorded on a Varian instrument operating at 400 MHz. ¹H NMR spectra were recorded on a Varian instrument operating at 400 MHz. ¹H-NMR spectra were obtained as CDCl₃, CD₃OD, or d₆-DMSO solutions (reported in ppm), using tetramethylsilane (0.00 ppm) or residual solvent (CDCl₃: 7.25 ppm; CD₃OD: 3.31 ppm; D₂O: 4.79 ppm; d₆-DMSO: 2.50 ppm) as the reference standard. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

### BIOLOGICAL EXAMPLES

### Example A

### MMP-13 Assay

The MMP-13 assay (C. Graham Knight, Frances Willenbrock, and Gillian Murphy (1992) FEBS Letters 296 (3), 263-266) is based on intramolecular fluorescence resonance energy transfer for detection of the activity of Matrix Metalloproteinase 13 (MMP-13) using a quenched-fluorogenic substrate. The cleavage reaction is detected continuously by the elevation of the fluorescence due to release from quenching. The fluorescence of the product increases in proportion to progress of the cleavage reaction.

MMP-13 (baculovirus expressed full-length protein) and inhibitor in assay buffer (50 mM HEPES, pH 7.4, 100 mM NaCl, 5 mM CaCl₂, 0.005% Brij-35, final DMSO concentration 1% (v/v)) are pre-incubated for 10 minutes at ambient temperature. MMP-2/MMP-7 Substrate, Mca-PLGL-Dap(Dnp)-AR-NH₂(AnaSpec, Inc.), is added and the plates are monitored over the length of the assay (20 minutes) for change in fluorescence (excitation 325, emission 395). inhibitors are also tested in the same buffer with 0.5% HSA (Human Serum Albumin, Sigma) to assess shift in activity in the presence of serum albumin.

When tested in this assay, compounds of Examples 1-35 had an IC₅₀ of less than 10 μM. In addition, the compounds of Examples 1-35 exhibited less than a 20-fold shift in activity in the presence of 0.5% HSA.

### PREPARATIVE EXAMPLES

### Example 1

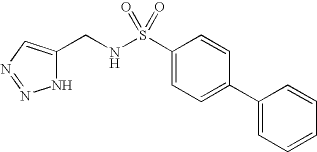

### N-((1H-1,2,3-triazol-5-yl)methyl)biphenyl-4-sulfonamide

Step A: tert-Butyl prop-2-ynylcarbamate: A solution of propargylamine (5.00 g, 90.8 mmol) and Boc₂O (18.8 g, 86.2 mmol) in DCM (200 mL) was stirred for 12 hours. The mixture was washed with dilute aqueous HCl, and the organic layer was dried (Na₂SO₄), filtered, and concentrated in vacuo. The resulting oil crystallized upon standing to give 12.0 g (90%) of the title compound. ¹H NMR (400 MHz, CDCl₃) δ 4.78 (br s, 1H), 3.92 (s, 2H), 2.22 (s, 1H), 1.46 (s, 9H).

Step B: tert-Butyl(1H-1,2,3-triazol-5-yl)methylcarbamate: A solution of tert-butyl prop-2-ynylcarbamate (2.00 g, 12.9 mmol), azidotrimethylsilane (2.60 mL, 19.3 mmol), and CuI (123 mg, 0.65 mmol) in 9:1 DMF/MeOH (25 mL) was heated at 95° C. in a sealed vessel for 12 hours. The mixture was concentrated in vacuo, the resulting oil was diluted with DCM, and the undissolved solids were filtered off. The filtrate was concentrated in vacuo to give 1.80 g of the title compound. MS APCI (+) m/z 199 detected. NMR (400 MHz, CDCl₃) δ 7.65 (s, 1H), 5.37 (br s, 1H), 4.44 (d, J=5.9 Hz, 2H), 1.45 (s, 9H).

Step C: (1H-1,2,3-triazol-5-yl)methanamine hydrochloride: A solution of tert-butyl (1H-1,2,3-triazol-5-yl)methylcarbamate (1.80 g, 9.1 mmol) in 4.0M HCl in dioxane (40 mL) was stirred for 2 hours resulting in the formation of a precipitate. The mixture was concentrated in vacuo to ¼ volume, diluted with acetonitrile, and filtered to give 1.3 g (100%) of the title compound as a light yellow solid. ¹H NMR (400 MHz, DMSO-d₆) δ 8.60 (br s, 4H), 7.94 (s, 1H), 4.04 (m, 2H).

Step D: N-((1H-1,2,3-triazol-5-yl)methyl)biphenyl-4-sulfonamide: Biphenyl-4-sulfonyl chloride (80 mg, 0.32 mmol) was added to a solution of (1H-1,2,3-triazol-5-yl)methanamine hydrochloride (45 mg, 0.33 mmol) in pyridine (3.0 mL). After stirring for 12 hours at ambient temperature, the mixture was partitioned between dilute aqueous HCl and DCM. The separated DCM layer was dried (Na₂SO₄), filtered, and concentrated in vacuo. The resulting solid was dissolved in isopropyl acetate (3 mL) with heating at 60° C., and then allowed to cool to ambient temperature with stirring to give a white crystalline precipitate. The solid was filtered off and air-dried to give 17 mg (15%) of the title compound. MS APCI (−) m/z 313 detected. ¹H NMR (400 MHz, DMSO-d₆) δ 8.14 (m, 1H), 7.81-7.37 (m, 10H), 4.07 (m, 2H).

### Example 2

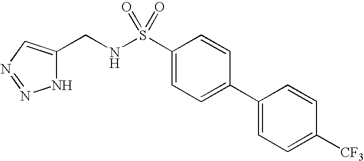

### N-((1H-1,2,3-triazol-5-yl)methyl)-4′-(trifluoromethyl)biphenyl-4-sulfonamide

Prepared from 4′-(trifluoromethyl)biphenyl-4-sulfonyl chloride (100 mg; 0.32 mmol) and (1H-1,2,3-triazol-5-yl)methanamine hydrochloride (Example 1, Step C; 45 mg, 0.33 mmol) according to the method of Example 1 to give 21 mg (16%) of the title compound. MS APCI (−) m/z 381 detected. NMR (400 MHz, DMSO-d₆) δ 8.20 (t, J=5.9 Hz, 1H), 7.88 (m, 8H), 7.53 (br s, 1H), 4.08 (d, J=6.2 Hz, 2H).

### Example 3

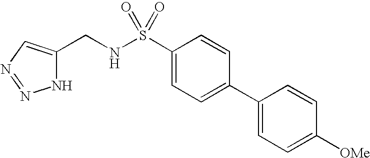

### N-((1H-1,2,3-triazol-5-yl)methyl)-4′-(methoxy)biphenyl-4-sulfonamide

Prepared from (1H-1,2,3-triazol-5-yl)methanamine hydrochloride (Example 1, Step C; 40 mg, 0.30 mmol) and 4′-(methoxy)biphenyl-4-sulfonyl chloride according to the method of Example 1 to provide title compound as a beige solid (23 mg, 22%). MS APCI (−) m/z 343 detected. NMR (400 MHz, CD₃OD) δ 7.85 (d, J=8.6 Hz, 2H), 7.74 (d, J=8.6 Hz, 2H), 7.63 (d, J=9.0 Hz, 2H), 7.55 (s, 1H), 7.04 (d, J=9.0 Hz, 2H), 4.24 (s, 2H), 3.85 (s, 3H).

### Example 4

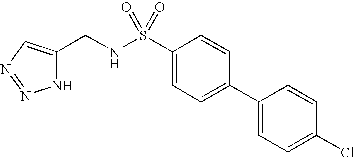

### N-((1H-1,2,3-triazol-5-yl)methyl)-4′-(chloro)biphenyl-4-sulfonamide

Prepared from (1H-1,2,3-triazol-5-yl)methanamine hydrochloride (Example 1, Step C; 40 mg, 0.30 mmol) and 4′-(chloro)biphenyl-4-sulfonyl chloride (94 mg, 0.33 mmol) according to the method of Example 1 to provide the title compound as a white solid (24 mg, 23%). MS APCI (−) m/z 347 detected. NMR (400 MHz, DMSO-d₆) δ 8.22-7.50 (m, 9H), 4.12 (m, 2H).

### Example 5

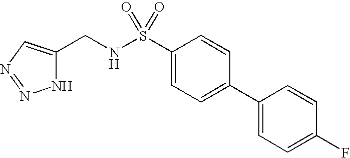

### N-((1H-1,2,3-triazol-5-yl)methyl)-4′-(fluoro)biphenyl-4-sulfonamide

Prepared from (1H-1,2,3-triazol-5-yl)methanamine hydrochloride (Example 1, Step C; 40 mg, 0.30 mmol) and 4′-(fluoro)biphenyl-4-sulfonyl chloride according to the method of Example 1 to provide the title compound as a white solid (30 mg, 30%). MS APCI (−) m/z 331 detected. ¹H NMR (400 MHz, DMSO-d₆) δ 8.20 (br s, 1H), 7.85-7.33 (m, 9H), 4.11 (d, J=5.9 Hz, 2H).

### Example 6

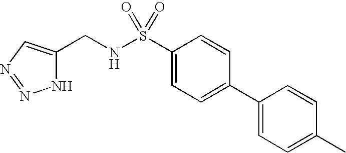

### N-((1H-1,2,3-triazol-5-yl)methyl)-4′-(methyl)biphenyl-4-sulfonamide

Prepared from (1H-1,2,3-triazol-5-yl)methanamine hydrochloride (Example 1, Step C; 40 mg, 0.30 mmol) and 4′-(methyl)biphenyl-4-sulfonyl chloride (89 mg, 0.33 mmol) according to the method of Example 1 to provide the title compound as a white solid (12 mg, 12%). MS APCI (−) m/z 327 detected. NMR (400 MHz, DMSO-d₆) δ 8.16-7.32 (m, 9H), 4.10 (d, J=5.9 Hz, 2H), 2.39 (s, 3H).

### Example 7

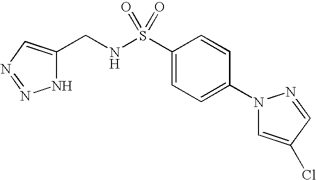

### N-((1H-1,2,3-triazol-5-yl)methyl)-4-(4-chloro-1H-pyrazol-1-yl)benzenesulfonamide

Prepared from 4-(4-chloro-1H-pyrazol-1-yl)benzene-1-sulfonyl chloride

(prepared according to Journal of the Chemical Society Section C: Organic 1968, 9, 1120) (88 mg, 0.32 mmol) and (1H-1,2,3-triazol-5-yl)methanamine hydrochloride (prepared according to Example 1, Step C; 45 mg, 0.33 mmol) according to the method of Example 1 to give 12 mg (11%) of the title compound. MS APCI (−) m/z 337 detected. ¹H NMR (400 MHz, DMSO-d₆) δ 8.94 (m, 1H), 8.00 (m, 7H), 4.13 (m, 2H).

### Example 8

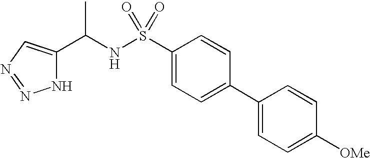

### N-(1-(1H-1,2,3-triazol-5-yl)ethyl)-4′-methoxybiphenyl-4-sulfonamide

Step A: tert-Butyl but-3-yn-2-yl(4′-methoxybiphenyl-4-ylsulfonyl)carbamate: Diisopropyl azodicarboxylate (DIAD) (0.26 mL, 1.32 mmol) was dropped into a solution of tert-butyl 4′-methoxybiphenyl-4-ylsulfonylcarbamate (J. Med. Chem. 2002, 45, 5628) (400 mg, 1.10 mmol), 3-butyn-2-ol (93 mg, 1.32 mmol), and Ph₃P (346 mg, 1:32 mmol) in THF (4 mL). After stirring for 12 hours at ambient temperature, the mixture was concentrated in vacuo and chromatographed (SiO₂) using 1:1 DCM/Hexane as eluent to give 430 mg (94%) of the title compound as a colorless gum. MS APCI (−) m/z 314 (loss of Boc) detected.

Step B: N-(but-3-yn-2-yl)-4′-methoxybiphenyl-4-sulfonamide: TFA (2 mL) was added to a solution of tert-butyl but-3-yn-2-yl(4′-methoxybiphenyl-4-ylsulfonyl)carbamate (430 mg, 1.03 mmol) in DCM (20 mL). After stirring for 12 hours, the mixture was diluted with toluene and concentrated in vacuo to give 317 mg (97%) of the title compound as an off-white solid. MS APCI (−) m/z 314 detected.

Step C: N-(1-(1H-1,2,3-triazol-5-yl)ethyl)-4′-methoxy biphenyl-4-sulfonamide: A mixture of N-(but-3-yn-2-yl)-4′-methoxybiphenyl-4-sulfonamide (317 mg, 1.01 mmol), CuI (10 mg, 0.05 mmol), and azidotrimethylsilane (0.16 mL, 1.21 mmol) in DMF (1.8 mL) and MeOH (0.2 mL) was heated at 90° C. for 12 hours. The mixture was concentrated in vacuo and chromatographed (SiO₂) using DCM followed by 1:1 DCM/Et₂O as eluent to give 140 mg (39%) of the title compound as a white solid. MS APCI (−) m/z 357 detected. ¹H NMR (400 MHz, DMSO-d₆) δ 8.21 (br s, 1H), 7.77 (s, 4H), 7.69 (d, J=9.0 Hz, 2H), 7.44 (br s, 1H), 7.06 (d, J=9.0 Hz, 2H), 4.56 (m, 1H), 3.82 (s, 3H), 1.31 (d, J=7.0 Hz, 3H).

### Example 9

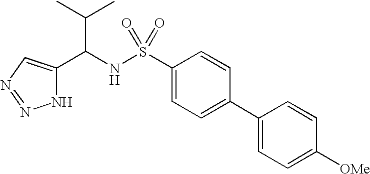

### 4′-Methoxy-N-(2-methyl-1-(1H-1,2,3-triazol-5-yl)propyl)biphenyl-4-sulfonamide

Prepare from 4-methylpent-1-yn-3-ol (130 mg, 1.32 mmol) and tert-butyl 4′-methoxybiphenyl-4-ylsulfonylcarbamate according to the method of Example 8 to give the title compound as a white solid (49 mg, 20%). MS APCI (−) m/z 385 detected. NMR (400 MHz, DMSO-d₆) δ 8.15 (d, J=12.1 Hz, 1H), 7.65 (m, 6H), 7.35 (s, 1H), 7.05 (d, J=8.6 Hz, 2H), 4.26 (m, 1H), 3.81 (s, 3H), 1.95 (m, 1H), 0.83 (d, J=6.6 Hz, 3H), 0.72 (d, J=6.6 Hz, 3H).

### Example 10

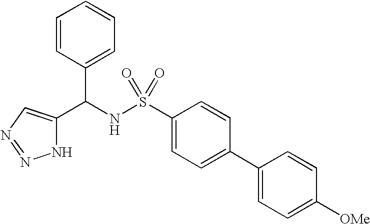

### 4′-Methoxy-N-(phenyl(1H-1,2,3-triazol-5-yl)methyl)biphenyl-4-sulfonamide

Prepared from 1-phenylprop-2-yn-1-ol (175 mg, 1.32 mmol) and tert-butyl 4′-methoxybiphenyl-4-ylsulfonylcarbamate according to the method of Example 8 to provide the title compound as a white solid (4 mg, 1.7%). MS APCI (−) m/z 419 detected. ¹H NMR (400 MHz, CD₃OD) δ 7.65 (d, J=8.6 Hz, 2H), 7.52 (m, 4H), 7.44 (s, 1H), 7.15 (m, 5H), 6.99 (d, J=9.0 Hz, 2H), 5.75 (s, 1H), 3.81 (s, 3H).

### Example 11

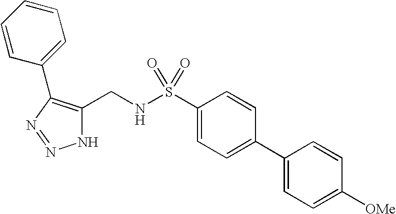

4′-Methoxy-N-((4-phenyl-1H-1,2,3-triazol-5-yl)methyl)biphenyl-4-sulfonamide

Step A: tert-Butyl 4′-methoxy biphenyl-4-yl)sulfonyl(3-phenylprop-2-ynyl)carbamate: Diisopropyl azodicarboxylate (DIAD) (0.26 mL, 1.32 mmol) was added dropwise to a solution of tert-butyl 4′-methoxybiphenyl-4-ylsulfonylcarbamate (J. Med. Chem. 2002, 45, 5628) (400 mg, 1.10 mmol), the 3-phenyl-2-propyn-1-ol (175 mg, 1.32 mmol), and Ph₃P (346 mg, 1.32 mmol) in THF (4 mL). After stirring for 12 hours, the mixture was concentrated in vacuo and chromatographed (SiO₂) using 1:1 DCM/Hexane as eluent to give 412 mg (78%) of the title compound as a colorless gum. MS APCI (−) m/z 376 (loss of Boc) detected.

Step B: 4′-Methoxy-N-(3-phenylprop-2-ynyl)biphenyl-4-sulfonamide: LiOH (83 mg, 3.45 mmol) in H₂O (1 mL) was added to a solution of tert-butyl 4′-methoxybiphenyl-4-ylsulfonyl(3-phenylprop-2-ynyl)carbamate (412 mg, 0.86 mmol) in MeOH (5 mL) and THF (2 mL). After stirring for 12 hours, the mixture was concentrated in vacuo and partitioned between DCM and dilute aqueous HCl. The separated DCM layer was dried (Na₂SO₄), filtered, and concentrated in vacuo to give 260 mg (80%) of the title compound. MS APCI (−) m/z 376 detected.

Step C: 4′-Methoxy-N-((4-phenyl-1H-1,2,3-triazol-5-yl)methyl)biphenyl-4-sulfonamide: A mixture of 4′-methoxy-N-(3-phenylprop-2-ynyl)biphenyl-4-sulfonamide (248 mg, 0.66 mmol), TMSN₃ (1.32 mL, 9.86 mmol) in DMF (1 mL) and MeOH (0.4 mL) was heated at 90° C. for 12 hours. The mixture was concentrated in vacuo and chromatographed (SiO₂) using DCM followed by 1:1 DCM/Et₂O as eluent to give 8 mg (2.9%) of the title compound as a gum. MS APCI (−) m/z 419 detected. ¹H NMR (400 MHz, CDCl₃) δ 7.82 (m, 2H), 7.59-7.39 (m, 10H), 7.00 (m, 2H), 4.47 (d, J=5.9 Hz, 2H), 3.86 (s, 3H).

### Example 12

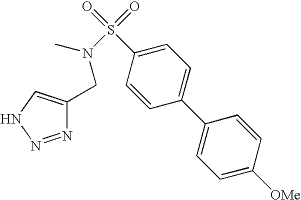

### N-((1H-1,2,3-triazol-4-yl)methyl)-4′-methoxy-N-methylbiphenyl-4-sulfonamide

Step A: 4′-ethoxy-N-(prop-2-ynyl)biphenyl-4-sulfonamide: Propargyl amine (0.970 mL, 14.2 mmol) and 4′-methoxybiphenyl-4-sulfonyl chloride (4.00 g, 14.2 mmol) were dissolved in pyridine (0.4M) and stirred for 16 hours at ambient temperature. After concentrating in vacuo, the resulting solid was suspended in EtOAc and byproducts were removed by filtration. The filtrate was concentrated to provide 4′-methoxy-N-(prop-2-ynyl)biphenyl-4-sulfonamide as a beige solid (87%).

Step B: 4′-Methoxy-N-methyl-N-(prop-2-ynyl)biphenyl-4-sulfonamide: Potassium carbonate (0.344 mg, 2.49 mmol) and methyl iodide (0.104 mL, 1.66 mmol) were added to a solution of 4′-methoxy-N-(prop-2-ynyl)biphenyl-4-sulfonamide (250 mg, 0.830 mmol) in DMF (0.2M). After stirring at ambient temperature for 16 hours, the reaction mixture was diluted in EtOAc and washed with water and brine. The organic layer was dried over MgSO₄ and concentrated in vacuo, and the resulting yellow solid was washed with Et₂O to afford 4′-methoxy-N-methyl-N-(prop-2-ynyl)biphenyl-4-sulfonamide (77%).

Step C: N-((1H-1,2,3-triazol-4-yl)methyl)-4′-methoxy-N-methylbiphenyl-4-sulfonamide: 4′-methoxy-N-methyl-N-(prop-2-ynyl)biphenyl-4-sulfonamide (200 mg, 0.634 mmol) was dissolved in a mixture of 9:1 DMF/MeOH (0.5M). To this was added trimethylsilyl azide (0.126 mL, 0.951 mmol) and Cu(I) I (6.04 mg, 0.032 mmol). The reaction mixture was heated to 100° C. for 16 hours. The Cu salts were removed by filtration and washing with EtOAc/MeOH. The filtrate was then washed with water and brine, dried over MgSO₄ and concentrated in vacuo. The residue was treated with DCM, and product crashed out and was filtered to afford N—(OH-1,2,3-triazol-4-yl)methyl)-4′-methoxy-N-methylbiphenyl-4-sulfonamide as a white solid (27%). MS APCI (−) m/z 357 detected. ¹H NMR (400 MHz, DMSO-d₆) δ 7.85 (m, 4H), 7.63 (br s, 1H), 7.73 (d, 2H), 7.08 (d, 2H), 4.33 (s, 2H), 3.82 (s, 3H), 2.66 (s, 3H).

### Example 13

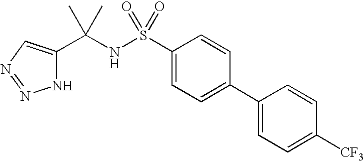

### N-(2-(1H-1,2,3-triazol-5-yl)propan-2-yl)-4′-(trifluoromethyl)biphenyl-4-sulfonamide

Step A: N-(2-Methylbut-3-yn-2-yl)-4′-(trifluoromethyl)biphenyl-4-sulfonamide: To a solution of 4′-(trifluoromethyl)biphenyl-4-sulfonyl chloride (304 mg, 0.95 mmol) in THF (4 mL) was added PS-DMAP (842 mg, 1.26 mmol) and the reaction mixture was swirled at ambient temperature for 10 minutes. The 2-methylbut-3-yn-2-amine (75 mg, 0.90 mmol) was then added. The reaction mixture was shaken at ambient temperature for 4 hours. PS-trisamine (300 mg, excess) was then added and the reaction mixture was shaken for 2 hours. MP-carbonate (300 mg, excess) was added and the reaction mixture was shaken for 2 hours. The resins were filtered off and the solvents were removed in vacuo. N-(2-methylbut-3-yn-2-yl)-4′-(trifluoromethyl)biphenyl-4-sulfonamide was obtained as a white solid (233 mg, 70%). MS APCI (−) m/z 366 detected.

Step B: N-(2-(1H-1,2,3-triazol-5-yl)propan-2-yl)-4′-(trifluoromethyl)biphenyl-4-sulfonamide: A solution of N-(2-methylbut-3-yn-2-yl)-4′-(trifluoromethyl)biphenyl-4-sulfonamide (220 mg, 0.60 mmol), azidotrimethylsilane (0.257 mL, 1.92 mmol), and CuI (5.7 mg, 0.03 mmol) in DMF/MeOH (1:1 v/v, 3 mL) was heated at 90° C. in a sealed vessel for 15 hours. The mixture was taken up into EtOAc (20 mL) and was washed with water and then 1N NaOH (aq) (15 mL). The basic layer was acidified with 1N HCl (aq) and the mixture was allowed to stand at ambient temperature for 30 minutes. The white solids were filtered off and dried to give N-(2-OH-1,2,3-triazol-5-yl)propan-2-yl)-4′-(trifluoromethyl)biphenyl-4-sulfonamide as a white solid (217 mg, 88%). MS APCI (−) m/z 411 detected. NMR (400 MHz, d₆-DMSO). MS APCI (−) m/z 409 detected. ¹H NMR (400 mHz. DMSO-d₆) δ 8.14 (s, 1H), 7.95 (s, 1H), 7.66-7.49 (m, 9H), 1.54 (s, 6H).

### Example 14

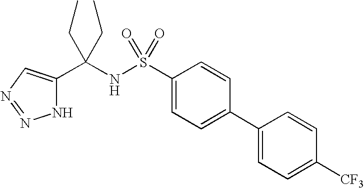

### N-(3-(1H-1,2,3-triazol-5-yl)pentan-3-yl)-4′-(trifluoromethyl)biphenyl-4-sulfonamide

Prepared according to the procedure described in Example 13 using 3-ethylpent-1-yn-3-amine and 4′-(trifluoromethyl)biphenyl-4-sulfonyl chloride. MS APCI (−) m/z 437 detected. ¹H. NMR (400 MHz, MeOH-d₄) δ 7.86 (d, J=8.6 Hz, 2H), 7.78 (d, J=8.6 Hz, 2H), 7.69-7.62 (m, 4H), 7.44 (br s, 1H), 2.21 (m, 2H), 2.01 (m, 2H), 0.76 (t, J=7.0 Hz, 6H).

### Example 15

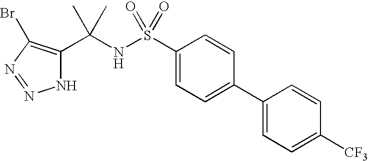

### N-(2-(4-bromo-1H-1,2,3-triazol-5-yl)propan-2-yl)-4′-(trifluoromethyl)biphenyl-4-sulfonamide

To a solution of N-(2-(1H-1,2,3-triazol-5-yl)propan-2-yl)-4′-(trifluoromethyl) biphenyl-4-sulfonamide (20 mg, 0.049 mmol) in MeOH/CHCl₃ (1:1 v/v, 4 mL) was added water (1 mL). Bromine (3 mL, excess) was added in three equal 1 mL portions over 40 hours while the reaction mixture was heated at 50° C. for 40 hours. To the reaction mixture was added EtOAc (15 mL), and the organics were washed with saturated NaHCO₃ (aq) and then saturated Na₂S₂O₃ (aq). The aqueous phase was dried (MgSO₄) and the solvents were removed in vacuo. To the white solid residue was added DCM (4 mL) and the solvent was decantered off to provide N-(2-(4-bromo-1H-1,2,3-triazol-5-yl)propan-2-yl)-4′-(trifluoromethyl)biphenyl-4-sulfonamide as a white solid (4 mg, 17%). MS APCI (−) m/z 487 and 489 detected.

### Example 16

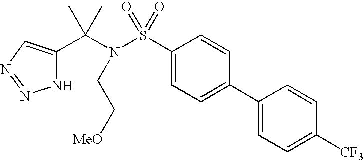

### N-(2-(1H-1,2,3-triazol-5-yl)propan-2-yl)-4′-(2-methoxyethyl)-4′-(trifluoromethyl)biphenyl-4-sulfonamide

Step A: N-(2-methylbut-3-yn-2-yl)-4′-(trifluoromethyl)biphenyl-4-sulfonamide: To a solution of 2-methylbut-3-yn-2-amine (250 mg, 3.01 mmol) in pyridine (7 mL) was added 4′-(trifluoromethyl)biphenyl-4-sulfonyl chloride (1.061 g, 3.31 mmol) in small portions over 5 minutes with no external cooling (exothermic). The reaction mixture was stirred at ambient temperature for 14 hours. The reaction mixture was diluted with DCM (100 mL), and the pyridine was extracted with 1N HCl (aq). The DCM layer was washed with 1N NaOH (aq) and brine. After drying the organic layer (MgSO₄), the solvent was removed in vacuo. N-(2-methylbut-3-yn-2-yl)-4′-(trifluoromethyl)biphenyl-4-sulfonamide (1.03 g, 93%) was isolated as a pale yellow crystalline solid. MS APCI (−) m/z 366 detected.

Step B: N-(2-methoxyethyl)-N-(2-methylbut-3-yn-2-yl)-4′-(trifluoromethyl)biphenyl-4-sulfonamide: To a solution of the N-(2-methylbut-3-yn-2-yl)-4′-(trifluoromethyl)biphenyl-4-sulfonamide (200 mg, 0.54 mmol) in DMF (4 mL) was added potassium carbonate (376 mg, 2.72 mmol) and 1-bromo-2-methoxyethane (54 μL, 0.57 mmol). The reaction mixture was stirred at 90° C. for 14 hours. A further portion of the 1-bromo-2-methoxyethane (20 μL, 0.21 mmol) was added, and the reaction mixture was heated at 90° C. for a further 5 hours. After cooling, the reaction mixture was diluted with EtOAc (15 mL), and the organics were washed with saturated NaHCO₃ (aq) and brine. After drying (MgSO₄), the solvents were removed in vacuo to give N-(2-methoxyethyl)-N-(2-methylbut-3-yn-2-yl)-4′-(trifluoromethyl)biphenyl-4-sulfonamide as a pale yellow oil (230 mg, 99%). This material was used without need for further purification. MS APCI (+) m/z 426 detected.

Step C: N-(2-(1H-1,2,3-triazol-5-yl)propan-2-yl)-N-(2-methoxyethyl)-4′-(trifluoromethyl)biphenyl-4-sulfonamide: A solution of N-(2-methoxy ethyl)-N-(2-methylbut-3-yn-2-yl)-4′-(trifluoromethyl)biphenyl-4-sulfonamide (230 mg, 0.54 mmol), TMSN₃ (0.239 mL, 1.78 mmol), and CuI (5 mg, 0.03 mmol) in DMF/MeOH (9:1 v/v, 3 mL) was heated at 90° C. in a sealed vessel for 15 hours. The mixture was concentrated and then 1N NaOH (aq) (15 mL) and DCM (20 mL) were added. The organic layer was washed again with 1N NaOH (15 mL), and the aqueous layers were combined. The aqueous layer was brought to pH 2 with 1N HCl (aq), and the product was extracted with EtOAc (20 mL). The organic phase was dried (MgSO₄), and the solvent was removed in vacuo. The crude residue was purified by PLC eluting with EtOAc to provide N-(2-(1H-1,2,3-triazol-5-yl)propan-2-yl)-N-(2-methoxyethyl)-4′-(trifluoromethyl)biphenyl-4-sulfonamide as a colorless oil (61 mg, 24%). MS APCI (+) m/z 469 detected. ¹H NMR (400 MHz, MeOH-d₄) δ 7.90-7.69 (m, 9H), 3.61 (m, 2H), 3.52 (m, 2H), 3.30 (s, 8H), 1.77 (s, 6H).

### Example 17

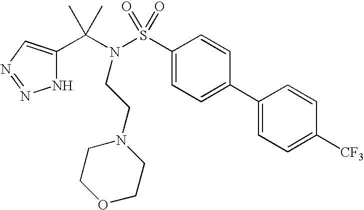

### N-(2-(1H-1,2,3-triazol-5-yl)propan-2-yl)-N-(2-morpholinoethyl)-4′-(trifluoromethyl)biphenyl-4-sulfonamide

Step A: N-(2-methylbut-3-yn-2-yl)-N-(2-morpholinoethyl)-4′-(trifluoromethyl)biphenyl-4-sulfonamide: To a solution of the N-(2-methylbut-3-yn-2-yl)-4′-(trifluoromethyl)biphenyl-4-sulfonamide (200 mg, 0.54 mmol) in DMF (4 mL) was added potassium carbonate (376 mg, 2.72 mmol) and 4-(2-chloroethyl)morpholine hydrochloride (78 mg, 0.57 mmol). The reaction mixture was stirred at 90° C. for 14 hours. A further portion of the 4-(2-chloroethyl)morpholine hydrochloride (78 mg, 0.57 mmol) was added, and the reaction mixture was heated at 90° C. for a further 5 hours. After cooling, the reaction mixture was diluted with EtOAc (15 mL), and the organics were washed with saturated NaHCO₃ (aq) and brine. After drying (MgSO₄), the solvents were removed in vacuo to give N-(2-methylbut-3-yn-2-yl)-N-(2-morpholinoethyl)-4′-(trifluoromethyl)biphenyl-4-sulfonamide as a pale yellow oil (260 mg, 99%). This material was used in the next step without further purification. MS APCI (+) m/z 481 detected.

Step B: N-(2-(1H-1,2,3-triazol-5-yl)propan-2-yl)-N-(2-morpholinoethyl)-4′-(trifluoromethyl)biphenyl-4-sulfonamide: A solution of N-(2-methylbut-3-yn-2-yl)-N-(2-morpholinoethyl)-4′-(trifluoromethyl)biphenyl-4-sulfonamide (260 mg, 0.54 mmol), TMSN₃ (0.239 mL, 1.78 mmol), and CuI (5 mg, 0.03 mmol) in DMF/MeOH (9:1 v/v, 3 mL) was heated at 90° C. in a sealed vessel for 15 hours. The mixture was concentrated and then 1N NaOH (aq) (15 mL) and DCM (20 mL) were added. The organic layer was washed again with 1N NaOH (15 mL), and the aqueous layers were combined. A white solid precipitated out from the aqueous phase on standing. The solids were filtered off, washed with water and dried to provide the title compound as a white solid (55 mg, 19%). MS APCI (+) m/z 524 detected. ¹H NMR (400 MHz, MeOH-d₄) δ 7.96 (d, J=9.0 Hz, 2H), 7.91 (d, J=9.0 Hz, 2H), 7.87 (d, J=9.0 Hz, 2H), 7.79 (d, J=9.0 Hz, 2H), 7.38 (s, 1H), 3.58 (m, 4H), 3.41 (m, 2H), 2.35 (m, 2H), 2.25 (m, 4H), 1.75 (s, 6H).

### Example 18

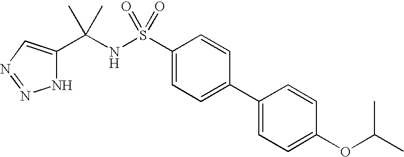

### N-(2-(1H-1,2,3-triazol-5-yl)propan-2-yl)-4′-isopropoxybiphenyl-4-sulfonamide

Step A: 4-Iodo-N-(2-methylbut-3-yn-2-yl)benzenesulfonamide: Prepared from 2-methylbut-3-yn-2-amine (5.00 g, 60.15 mmol) and 4-iodobenzene-1-sulfonyl chloride (18.20 g, 60.15 mmol) according to Example 8, Step A, to give 4-iodo-N-(2-methylbut-3-yn-2-yl)benzenesulfonamide as a white crystalline solid (11.0 g, 52%). MS APCI (−) m/z 348 detected.

Step B: 4′-Isopropoxy-N-(2-methylbut-3-yn-2-yl)biphenyl-4-sulfonamide: A mixture of 4-iodo-N-(2-methylbut-3-yn-2-yl)benzenesulfonamide (400 mg, 1.15 mmol), 4-isopropoxyphenylboronic acid (309 mg, 1.72 mmol), Na₂CO₃ (364 mg, 3.44 mmol) and Pd(PPh₃)₄ (132 mg, 0.15 mmol) were combined in DME (7 mL) and water (2 mL), and the mixture was degassed by bubbling through nitrogen for 10 minutes. The reaction mixture was sealed (sealed tube) under nitrogen, and the reaction mixture was heated at 85° C. for 18 hours. After cooling to ambient temperature, EtOAc (80 mL) was added, and the organics were washed with saturated NaHCO₃ (aq), brine, and dried (MgSO₄). The solvents were removed in vacuo to give an orange gum. The product was purified by silica gel plug eluting with 4:1 hexanes/EtOAc. 4′-isopropoxy-N-(2-methylbut-3-yn-2-yl)biphenyl-4-sulfonamide was isolated as a pale yellow solid (270 mg, 66%). MS APCI. (−) m/z 356 detected.

Step C: N-(2-(1H-1,2,3-triazol-5-yl)propan-2-yl)-4′-isopropoxybiphenyl-4-sulfonamide: Prepared from the product of Step B according to the method of Example 8, step B, to provide the title compound as a white solid (102 mg, 36%). MS APCI (−) m/z 399 detected. ¹H NMR (400 MHz, MeOH-d₄) δ 7.64-7.55 (m, 6H), 7.00 (d, J=9.0 Hz, 2H), 4.66 (m, 1H), 1.65 (s, 6H), 1.34 (d, J=6.2 Hz, 6H).

### Example 19

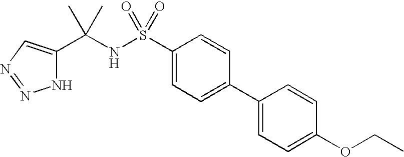

### N-(2-(1H-1,2,3-triazol-5-yl)propan-2-yl)-4′-ethoxybiphenyl-4-sulfonamide

Prepared according to the procedure of Example 18, Steps B and C using 4-ethoxyphenylboronic acid in Step B. The title compound was isolated as a white solid (30 mg, 18%). MS APCI (−) m/z 385 detected. NMR (400 MHz, MeOH-d₄) δ 7.65-7.58 (m, 7H), 7.00 (d, J=8.6 Hz, 2H), 4.08 (m, 2H), 1.65 (s, 6H), 1.39 (t, J=7.0 Hz, 3H).

### Example 20

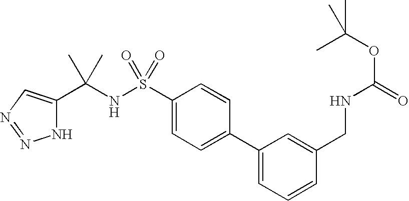

### tert-butyl(4′-(N-(2-(1H-1,2,3-triazol-5-yl)propan-2-yl)sulfamoyl)biphenyl-3-yl)methylcarbamate

Prepared according to the procedure of Example 18, Steps B and C, using 3-((tert-butoxycarbonyl)methyl)phenylboronic acid in Step B. The title compound was isolated as a white solid (150 mg, 34%). MS APCI (−) m/z 470 detected. ¹H NMR (400 MHz, MeOH-d₄) δ 7.68-7.32 (m, 9H), 4.30 (s, 2H), 3.35 (s, 1H), 1.66 (s, 6H), 1.46 (s, 9H).

### Example 21

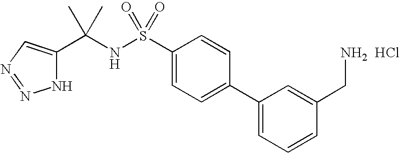

### N-(2-(1H-1,2,3-triazol-5-yl)propan-2-yl)-4′-(trifluoromethyl)biphenyl-4-sulfonamide hydrochloride

To a solution of tert-butyl(4′-(N-(2-(1H-1,2,3-triazol-5-yl)propan-2-yl)sulfamoyl)biphenyl-3-yl)methylcarbamate (137 mg, 0.29 mmol) in DCM (3 mL) was added a solution of HCl in dioxane (4.0M, 1 mL, 4.35 mmol). The solution was stirred at ambient temperature for 1 hour. The reaction mixture was concentrated in vacuo, and the residue was triturated with DCM (10 mL). The white solids were filtered off and dried. The title compound (92 mg, 78%) was isolated as a white solid. MS APCI (+) m/z 372 detected. ¹H NMR (400 MHz. MeOH-d₄) δ 8.01-7.51 (m, 9H), 4.22 (s, 2H), 1.67 (s, 6H).

### Example 22

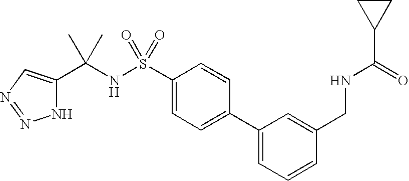

### N-((4′-(N-(2-(1H-1,2,3-triazol-5-yl)propan-2-yl)sulfamoyl)biphenyl-3-yl)methyl)cyclopropanecarboxamide

To a solution of N-(2-(1H-1,2,3-triazol-5-yl)propan-2-yl)-3′-(aminomethyl)biphenyl-4-sulfonamide hydrochloride (Example 21; 20 mg, 0.049 mmol) in THF/water (10:1 v/v, 4 mL) was added triethylamine (0.02 mL, 0.147 mmol) and resin bound cyclopropane carboxylic acid tetrafluorophenyl (TFP) ester (38 mg, 0.049 mmol, 1.29 mmol/g loading). The reaction mixture was swirled at ambient temperature for 14 hours. The resin was filtered off and washed with THF (20 mL). The solvents were removed in vacuo, and the residue was taken up into DCM (20 mL) and washed with 1N HCl (aq) (15 mL). The organic phase was washed with brine and then dried (MgSO₄). The solvent was removed in vacuo to provide the title compound (9 mg, 42%) as a colorless gum. MS APCI (−) m/z 438 detected. ¹H NMR (400 MHz, MeOH-d₄) δ 7.68-7.33 (m, 9H), 4.45 (s, 2H), 1.66-1.60 (m, 7H), 0.89 (m, 2H), 0.78 (m, 1H).

### Example 23

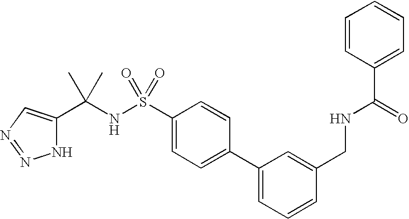

### N-((4′-(N-(2-(1H-1,2,3-triazol-5-yl)propan-2-yl)sulfamoyl)biphenyl-3-yl)methyl)benzamide

To a solution of N-(2-(1H-1,2,3-triazol-5-yl)propan-2-yl)-3′-(aminomethyl)biphenyl-4-sulfonamide hydrochloride (Example 21; 20 mg, 0.049 mmol) in THF/water (10:1 v/v, 4 mL) was added triethylamine (0.02 mL, 0.147 mmol) and resin bound benzoic acid tetrafluorophenyl (TFP) ester (39 mg, 0.049 mmol, 1.26 mmol/g loading). The reaction mixture was swirled at ambient temperature for 14 hours. The resin was filtered off and washed with THF (20 mL). The solvents were removed in vacuo, and the residue was taken up into DCM (20 mL) and washed with 1N HCl (aq) (15 mL). The organic phase was washed with brine and then dried (MgSO₄). The solvent was removed in vacuo to provide the title compound (9 mg, 39%) as a white solid. MS APCI (−) m/z 474 detected. ¹H NMR (400 MHz, MeOH-d₄) δ 7.88-7.41 (m, 14H), 4.66 (s, 2H), 1.65 (s, 6H).

### Example 24

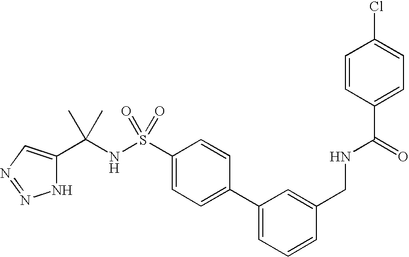

### N-((4′-(N-(2-(1H-1,2,3-triazol-5-yl)propan-2-yl)sulfamoyl)biphenyl-3-yl)methyl)-4-chlorobenzamide

Prepared according to the procedure of Example 23, using resin bound 4-chlorobenzoic acid tetrafluorophenyl (TFP) ester. MS APCI (−) m/z 509 detected. ¹H NMR (400 MHz, MeOH-d₄) δ 7.87-7.42 (m, 13H), 4.65 (s, 2H), 1.65 (s, 6H).

### Example 25

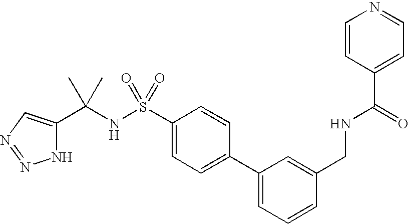

### N-((4′-(N-(2-(1H-1,2,3-triazol-5-yl)propan-2-yl)sulfamoyl)biphenyl-3-yl)methyl)isonicotinamide

Prepared according to the procedure of Example 23, using resin bound isonicotinic acid (TFP) ester. MS APCI (+) m/z 477 detected. ¹H NMR (400 MHz, MeOH-d₄) δ 8.70-7.43 (m, 13H), 4.58 (s, 2H), 1.65 (s, 6H).

### Example 26

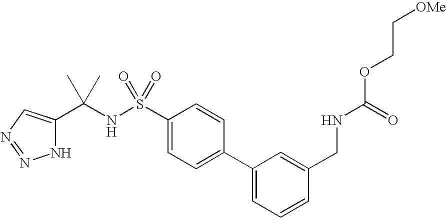

### 2-Methoxy ethyl(4′-(N-(2-(1H-1,2,3-triazol-5-yl)propan-2-yl)sulfamoyl)biphenyl-3-yl)methylcarbamate

To a solution of N-(2-(1H-1,2,3-triazol-5-yl)propan-2-yl)-3′-(aminomethyl)biphenyl-4-sulfonamide hydrochloride (35 mg, 0.0858 mmol) in THF (5 mL) and water (0.2 mL) was added triethylamine (0.0359 mL, 0.257 mmol) and 2-methoxyethyl carbonochloridate (0.00991 mL, 0.0858 mmol). The reaction was stirred at ambient temperature for 5 hours. EtOAc (20 mL) was added, and the organics were washed with water, brine and dried (MgSO₄). The solvents were removed in vacuo to give a colorless oil. The residue was purified by preparative liquid chromatography, eluting with EtOAc, to provide the title compound as a colorless oil (6 mg, 14.8% yield). MS APCI (−) m/z 471 detected. ¹H NMR (400 MHz, MeOH-d₄) δ 7.95-7.35 (m, 9H), 4.36 (s, 2H), 4.20 (m, 2H), 3.59 (m, 2H), 3.36 (s, 3H), 1.66 (s, 6H).

### Example 27

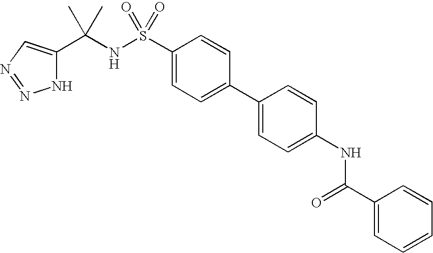

### N-(4′-(N-(2-(1H-1,2,3-triazol-5-yl)propan-2-yl)sulfamoyl)biphenyl-4-yl)benzamide

To a solution of N-(4′-(N-(2-methylbut-3-yn-2-yl)sulfamoyl)biphenyl-4-yl)benzamide (66 mg, 0.158 mmol) in DMF/MeOH (9:1 v/v, 3 mL) was added copper (1) iodide (2 mg, 0.011 mmol) and azidotrimethylsilane (0.211 mL, 1.57 mmol). The reaction was heated at 85° C. for 18 hours. The reaction mixture was concentrated in vacuo, and the residue was purified by reverse phase HPLC to provide the title compound as a white solid (10 mg, 14%) as a white solid. MS APCI (−) m/z 461 detected. ¹H NMR (400 MHz, MeOH-d₄) δ 7.93-7.49 (m, 14H), 1.63 (s, 6H).

### Example 28

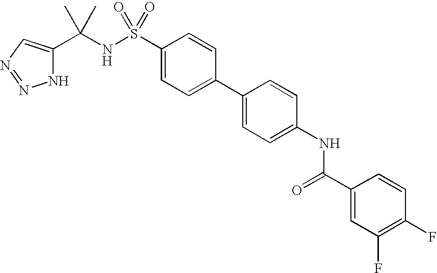

### N-(4′-(N-(2-(1H-1,2,3-triazol-5-yl)propan-2-yl)sulfamoyl)biphenyl-4-yl)-3,4-difluorobenzamide

Prepared from 3,4-difluoro-N-(4′-(N-(2-methylbut-3-yn-2-yl)sulfamoyl) biphenyl-4-yl)benzamide (106 mg, 0.233 mmol) according to the method of Example 27 to provide the title compound as a white solid (26 mg, 22%). MS APCI (−) m/z 497 detected. ¹H NMR (400 MHz, MeOH-d₄) δ 7.94-7.37 (m, 12H), 1.62 (s, 6H).

### Example 29

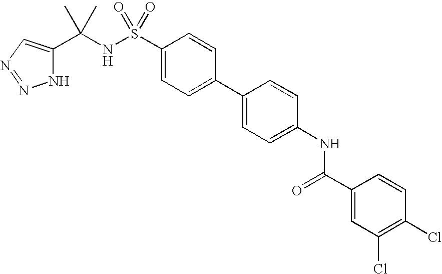

### N-(4′-(N-(2-(1H-1,2,3-triazol-5-yl)propan-2-yl)sulfamoyl)biphenyl-4-yl)-3,4-dichlorobenzamide

Prepared from 3,4-dichloro-N-(4′-(N-(2-methylbut-3-yn-2-yl)sulfamoyl) biphenyl-4-yl)benzamide (106 mg, 0.233 mmol) according to the method of Example 27 to provide the title compound as a white solid (15 mg, 15%). MS APCI (−) m/z 531. ¹H NMR (400 MHz, MeOH-d₄) δ 8.14-7.55 (m, 12H), 1.66 (s, 6H).

### Example 30

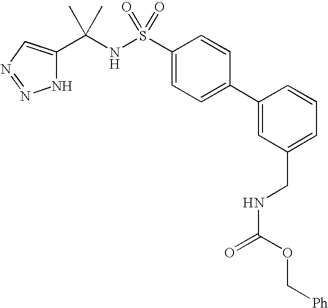

### Benzyl(4′-(N-(2-(1H-1,2,3-triazol-5-yl)propan-2-yl)sulfamoyl)biphenyl-3-yl)methylcarbamate

Prepared according to the procedure of Example 26, using benzyl chloroformate. MS APCI (+) m/z 506.0 detected. NMR (400 mHz. CD₃OD) δ 7.23-7.67 (m, 14H), 5.11 (s, 2H), 4.37 (s, 2H), 1.66 (s, 6H).

### Example 31

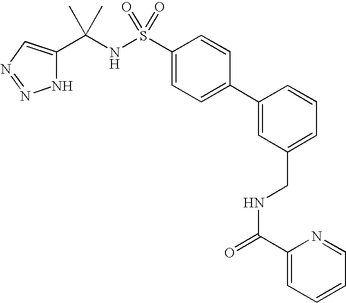

### N-((4′-(N-(2-(1H-1,2,3-triazol-5-yl)propan-2-yl)sulfamoyl)biphenyl-3-yl)methyl)picolinamide

Prepared according to the procedure of Example 23, using 2-pyridine PS-TFP (polystyrene bound tetrafluorophenol) ester. MS APCI (+) m/z 477.0 detected. NMR (400 mHz. CD₃OD) δ 8.64 (d, 1H, J=4.18 Hz), 8.12 (d, 1H, J=7.86 Hz), 7.96 (1, 1H, J=7.19 Hz), 7.62-7.70 (m, 5H), 7.51-7.59 (m, 3H), 7.40-7.48 (m, 2H), 4.69 (s, 2H), 1.65 (s,

### Example 32

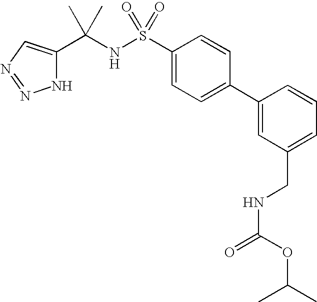

### Isopropyl(4′-(N-(2-(1H-1,2,3-triazol-5-yl)propan-2-yl)sulfamoyl)biphenyl-3-yl)methylcarbamate

Prepared according to the procedure of Example 26, using isopropyl chloroformate. MS APCI (+) m/z 458.0 detected. ¹H NMR (400 mHz. CDCl₃) δ 7.67-7.72 (m, 2H), 7.29-7.55 (m, 7H), 5.72 (br s, 1H, NH), 5.14 (br s, 1H, NH), 4.89-5.03 (m, 1H), 4.41 (d, 2H, J=6.35 Hz), 1.70 (s, 6H), 1.25 (d, 2H, J=6.28 Hz).

### Example 33

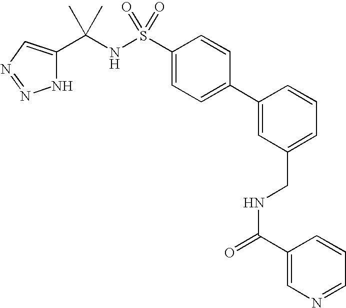

### N-((4′-(N-(2-(1H-1,2,3-triazol-5-yl)propan-2-yl)sulfamoyl)biphenyl-3-yl)methyl)nicotinamide

Prepared according to the procedure of Example 23, using 3-pyridine PS-TFP (polystyrene bound tetrafluorophenol) ester. MS APCI (+) m/z 477.0 detected. ¹H NMR (400 mHz. CD₃OD) δ 9.02-9.04 (m, 1H), 8.67-8.70 (m, 1H), 8.27-8.31 (m, 1H), 7.39-7.69 (m, 9H), 7.24 (s, 1H), 4.67 (s, 2H), 1.63 (s, 6H).

### Example 34

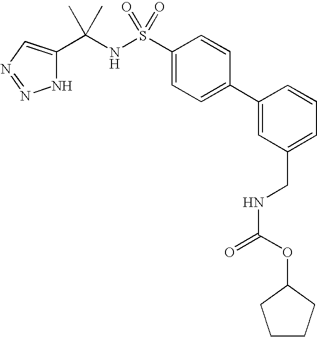

### Cyclopentyl(4′-(N-(2-(1H-1,2,3-triazol-5-yl)propan-2-yl)sulfamoyl)biphenyl-3-yl)methylcarbamate

Prepared according to the procedure of Example 26, using cyclopentyl chloroformate. MS APCI (+) m/z 484.0 detected. NMR (400 mHz. CD₃OD) δ 7.28-8.01 (m, 9H), 5.06 (m, 1H), 4.34 (s, 2H), 1.66 (s, 6H), 1.48-1.89 (m, 8H).

### Example 35

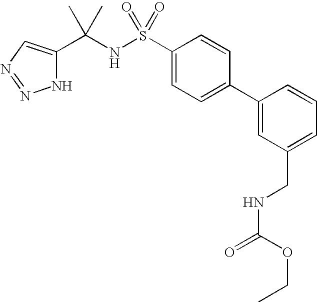

### Ethyl(4′-(N-(2-(1H-1,2,3-triazol-5-yl)propan-2-yl)sulfamoyl)biphenyl-3-yl)methylcarbamate

Prepared according to the procedure of Example 26, using ethyl chloroformate. MS APCI (+) m/z 443.9 detected. ¹H NMR (400 mHz. CD₃OD) δ 7.31-7.68 (m, 9H), 4.35 (s, 2H), 4.11 (m, 2H), 1.66 (s, 6H), 1.25 (m, 3H).

### Example 36

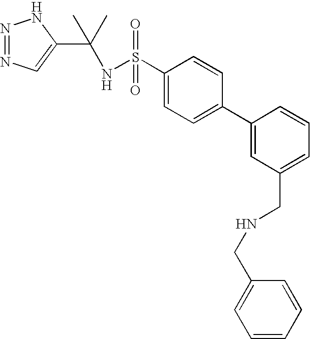

### N-(2-(1H-1,2,3-triazol-5-yl)propan-2-yl)-3′-((benzylamino)methyl)biphenyl-4-sulfonamide

N-(2-(1H-1,2,3-triazol-5-yl)propan-2-yl)-3′-(aminomethyl)biphenyl-4-sulfonamide hydrochloride (Example 21, 50.0 mg, 0.123 mmol) was dissolved in DMF (2 mL) and free-based using DIEA (42.7 uL, 0.245 mmol, 2.00 equivalents). Benzaldehyde (12.5 uL, 0.123 mmol, 1.00 equivalent), 4A molecular sieves (100 mg), and NaBH(OAc)₃ (39.0 mg, 0.184 mmol, 1.50 equivalents) were added to this reaction mixture. After stirring at room temperature for 4 hours, the reaction was incomplete and resubjected with NaBH(OAc)₃ (39.0 mg, 0.184 mmol, 1.50 equivalents) and HOAc (14.0 uL, 0.245 mmol, 2.00 equivalents). After stirring at room temperature overnight, the reaction mixture was diluted with water and EtOAc. The undissolved solid was filtered out. The filtrate was separated and washed with water (3×) and brine (1×). The aqueous layer was concentrated in vacuo, diluted with MeOH, and filtered to remove salts. The filtrate was combined with the organic extracts and purified by reverse phase HPLC using a 10-85% acetonitrile/H₂O gradient to afford N-(2-(1H-1,2,3-triazol-5-yl)propan-2-yl)-3′-((benzylamino) methyl)biphenyl-4-sulfonamide (2.9 mg, 5.1%). MS APCI (+) m/z 462.1 detected. ¹H NMR (400 mHz. CD₃OD) δ 7.20-7.94 (m, 14H), 3.79 (s, 2H), 3.73 (s, 2H), 1.62 (s, 6H).

The foregoing description is considered as illustrative only of the principles of the invention. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

The words “comprise,” “comprising,” “include,” “including,” and “includes” when used in this specification and in the following claims are intended to specify the presence of stated features, integers, components, or steps, but they do not preclude the presence or addition of one or more other features, integers, components, steps, or groups thereof.

What is claimed is:

1. A compound selected from Formula I: 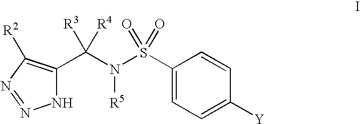
and enantiomers and salts thereof, wherein:
R² is H, Br, Cl, C₁-C₆ alkyl, Ar¹, or CH₂—Ar²;
R³ and R⁴ are independently H, C₁-C₆ alkyl, Ar³, CH₂—Ar⁴, or a 5-6 membered heteroaryl ring;
R⁵ is H, C₁-C₆ alkyl, (C₂-C₄ alkyl)OMe, or (C₂-C₄ alkyl)heterocyclyl;
Y is selected from the structures: 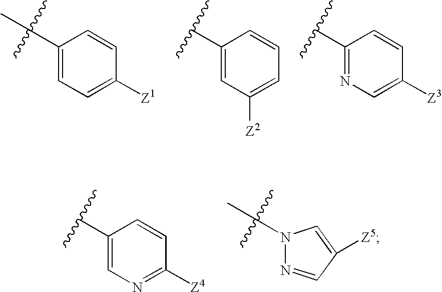
Z¹ is H, F, Cl, Br, CN, CF₃, C₁-C₆ alkyl, O—(C₁-C₆ alkyl), (C₁-C₆ alkyl)-OH, 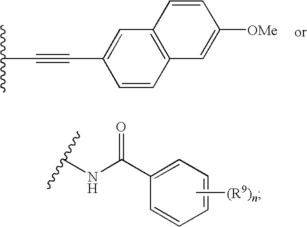
Z² is H, (C₁-C₃-alkyl)NH₂, CH₂NHR¹⁰, CH₂NHC(═O)OR⁷, or CH₂NHC(═O)R⁸;
Z³ is H, F, Cl, Br, CN, CF₃, C₁-C₆ alkyl, O—(C₁-C₆ alkyl), or (C₁-C₆ alkyl)-OH;
Z⁴ is H, CF₃, C₁-C₆ alkyl, or O—(C₁-C₆ alkyl);
Z⁵ is H, F, Cl, Br, CF₃, C₁-C₆ alkyl, or O—(C₁-C₆ alkyl);
R⁷ is C₁-C₆ alkyl, CH₂CH₂OMe, CH₂—Ar⁵, or a 3-6 membered cycloalkyl ring;
R⁸ is H, C₁-C₆ alkyl, CH₂-phenyl, a 3-6 membered cycloalkyl ring, C₆-C₁₀ aryl, or a 5-6-membered heteroaryl ring, wherein said aryl is optionally substituted with one to four R⁹ groups;
each R⁹ is independently F, Cl, CN, CF₃, C₁-C₆ alkyl, O—(C₁-C₆ alkyl), or (C₁-C₆ alkyl)-OH;
R¹⁰ is H or C₁-C₄ alkyl optionally substituted with a 5 or 6 membered aryl;
Ar¹, Ar², Ar³, Ar⁴ and Ar⁵ are each phenyl optionally and independently substituted with one or two groups independently selected from F, Cl, Br, I, C₁-C₃ alkyl, and O—(C₁-C₃ alkyl); and
n is 0, 1, 2, 3 or 4.

2. The compound of claim 1, wherein R⁵ is H, methyl, ethyl, CH₂CH₂OCH₃, or CH₂CH₂-(morpholin-4-yl).

3. The compound of claim 2, wherein R⁵ is H.

4. The compound of claim 2, wherein R² is H, Br, or phenyl.

5. The compound of claim 4, wherein R³ and R⁴ are independently H, phenyl, methyl, ethyl, isopropyl, benzyl, 2-pyridyl, 3-pyridyl or 4-pyridyl.

6. The compound claim 5, wherein R³ and R⁴ are H.

7. The compound claim 5, wherein R³ and R⁴ are methyl.

8. The compound claim 7, wherein Y is:

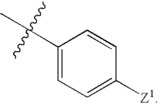

9. The compound of claim 8, wherein Z¹ is H, F, Cl, CN, CF₃, methyl, ethyl, isopropyl, OCH₃, OCH₂CH₃, O—CH(CH₃)₂, CH₂OH, or 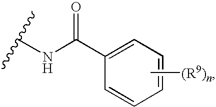
wherein each R⁹ is independently F, Cl, OCH₃, CH₂OH, CN or CF₃.

10. The compound of claim 9, wherein Z¹ is selected from the structures:

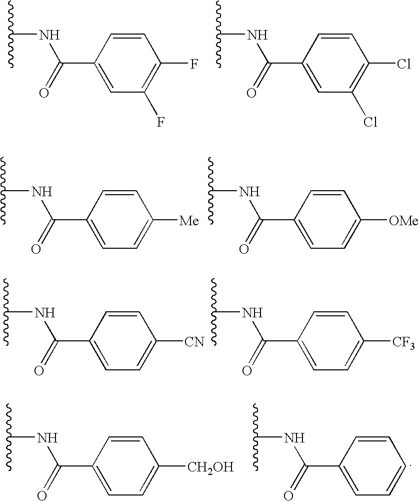

11. The compound of claim 7, wherein Y is:

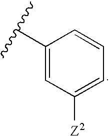

12. The compound of claim 11, wherein Z² is CH₂NH₂ or CH₂CH₂NH₂.

13. The compound of claim 11, wherein Z² is CH₂NHC(═O)OR⁷ and R⁷ is methyl, ethyl, isopropyl, t-butyl, cyclopentyl, CH₂CH₂OCH₃, or CH₂-phenyl.

14. The compound of claim 11, wherein Z² is CH₂NHC(═O)R⁸ and R⁸ is H, methyl, ethyl, isopropyl, t-butyl, cyclopropyl, cyclobutyl, cyclopentyl, cyclohexyl, 2-pyridyl, 3-pyridyl, 4-pyridyl, benzyl, naphthyl, phenyl, 4-fluorophenyl, 4-chlorophenyl, 4-methylphenyl, 4-methoxyphenyl, 4-(hydroxymethyl)phenyl, 4-cyanomethyl, or 4-(trifluoromethyl)phenyl.

15. The compound of claim 7, wherein Y is:

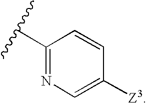

16. The compound of claim 15, wherein Z³ is H, F, Cl, Br, methyl, ethyl, propyl, isopropyl, OCH₃, OCH₂CH₃, O—CH(CH₃)₂, CH₂OH, CN or CF₃.

17. The compound of claim 7, wherein Y is:

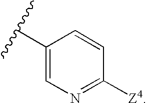

18. The compound of claim 17, wherein Z⁴ is H, methyl, ethyl, OCH₃, OCH₂CH₃, or CF₃.

19. The compound of claim 7, wherein Y is:

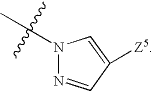

20. The compound of claim 19, wherein Z⁵ is H, F, Cl, Br, methyl, ethyl, OCH₃, OCH₂CH₃, or CF₃.

21. The compound of claim 1, selected from:
N-((1H-1,2,3-triazol-5-yl)methyl)biphenyl-4-sulfonamide;
N-((1H-1,2,3-triazol-5-yl)methyl)-4′-(trifluoromethyl)biphenyl-4-sulfonamide;
N-((1H-1,2,3-triazol-5-yl)methyl)-4′-(methoxy)biphenyl-4-sulfonamide;
N-((1H-1,2,3-triazol-5-yl)methyl)-4′-(chloro)biphenyl-4-sulfonamide;
N-((1H-1,2,3-triazol-5-yl)methyl)-4′-(fluoro)biphenyl-4-sulfonamide;
N-((1H-1,2,3-triazol-5-yl)methyl)-4′-(methyl)biphenyl-4-sulfonamide;
N-((1H-1,2,3-triazol-5-yl)methyl)-4-(4-chloro-1H-pyrazol-1-yl)benzenesulfonamide;
N-(1-(1H-1,2,3-triazol-5-yl)ethyl)-4′-methoxybiphenyl-4-sulfonamide;
4′-methoxy-N-(2-methyl-1-(1H-1,2,3-triazol-5-yl)propyl)biphenyl-4-sulfonamide;
4′-methoxy-N-(phenyl(1H-1,2,3-triazol-5-yl)methyl)biphenyl-4-sulfonamide;
4′-methoxy-N-((4-phenyl-1H-1,2,3-triazol-5-yl)methyl)biphenyl-4-sulfonamide;
N-((1H-1,2,3-triazol-4-yl)methyl)-4′-methoxy-N-methylbiphenyl-4-sulfonamide;
N-(2-(1H-1,2,3-triazol-5-yl)propan-2-yl)-4′-(trifluoromethyl)biphenyl-4-sulfonamide;
N-(3-(1H-1,2,3-triazol-5-yl)pentan-3-yl)-4′-(trifluoromethyl)biphenyl-4-sulfonamide;
N-(2-(4-bromo-1H-1,2,3-triazol-5-yl)propan-2-yl)-4′-(trifluoromethyl)biphenyl-4-sulfonamide;
N-(2-(1H-1,2,3-triazol-5-yl)propan-2-yl)-N-(2-methoxyethyl)-4′-(trifluoromethyl) biphenyl-4-sulfonamide;
N-(2-(1H-1,2,3-triazol-5-yl)propan-2-yl)-N-(2-morpholinoethyl)-4′-(trifluoromethyl)biphenyl-4-sulfonamide;
N-(2-(1H-1,2,3-triazol-5-yl)propan-2-yl)-4′-isopropoxybiphenyl-4-sulfonamide;
N-(2-(1H-1,2,3-triazol-5-yl)propan-2-yl)-4′-ethoxybiphenyl-4-sulfonamide;
tert-butyl(4′-(N-(2-(1H-1,2,3-triazol-5-yl)propan-2-yl)sulfamoyl)biphenyl-3-yl)methylcarbamate;
N-(2-(1H-1,2,3-triazol-5-yl)propan-2-yl)-4′-(trifluoromethyl)biphenyl-4-sulfonamide;
N-((4′-(N-(2-(1H-1,2,3-triazol-5-yl)propan-2-yl)sulfamoyl)biphenyl-3-yl)methyl)cyclopropanecarboxamide;
N-((4′-(N-(2-(1H-1,2,3-triazol-5-yl)propan-2-yl)sulfamoyl)biphenyl-3-yl)methyl)benzamide;
N-((4′-(N-(2-(1H-1,2,3-triazol-5-yl)propan-2-yl)sulfamoyl)biphenyl-3-yl)methyl)-4-chlorobenzamide;
N-((4′-(N-(2-(1H-1,2,3-triazol-5-yl)propan-2-yl)sulfamoyl)biphenyl-3-yl)methyl)isonicotinamide;
2-Methoxyethyl(4′-(N-(2-(1H-1,2,3-triazol-5-yl)propan-2-yl)sulfamoyl)biphenyl-3-yl)methylcarbamate;
N-(4′-(N-(2-(1H-1,2,3-triazol-5-yl)propan-2-yl)sulfamoyl)biphenyl-4-yl)benzamide;
N-(4′-(N-(2-(1H-1,2,3-triazol-5-yl)propan-2-yl)sulfamoyl)biphenyl-4-yl)-3,4-difluorobenzamide;
N-(4′-(N-(2-(1H-1,2,3-triazol-5-yl)propan-2-yl)sulfamoyl)biphenyl-4-yl)-3,4-dichlorobenzamide;
benzyl(4′-(N-(2-(1H-1,2,3-triazol-5-yl)propan-2-yl)sulfamoyl)biphenyl-3-yl)methylcarbamate;
N-((4′-(N-(2-(1H-1,2,3-triazol-5-yl)propan-2-yl)sulfamoyl)biphenyl-3-yl)methyl)picolinamide;
isopropyl(4′-(N-(2-(1H-1,2,3-triazol-5-yl)propan-2-yl)sulfamoyl)biphenyl-3-yl)methylcarbamate;
N-((4′-(N-(2-(1H-1,2,3-triazol-5-yl)propan-2-yl)sulfamoyl)biphenyl-3-yl)methyl)nicotinamide;
cyclopentyl(4′-(N-(2-(1H-1,2,3-triazol-5-yl)propan-2-yl)sulfamoyl)biphenyl-3-yl)methylcarbamate;
ethyl(4′-(N-(2-(1H-1,2,3-triazol-5-yl)propan-2-yl)sulfamoyl)biphenyl-3-yl)methylcarbamate;
N-(2-(1H-1,2,3-triazol-5-yl)propan-2-yl)-3′-((benzylamino)methyl)biphenyl-4-sulfonamide; and
salts thereof.

22. A pharmaceutical composition comprising a compound as claimed in claim 1.

23. A method of treating an MMP-mediated disease or disorder in a mammal, said method comprising administering to said mammal a therapeutically effective amount of a compound as claimed in claim 1,

24. The method of claim 23, wherein the MMP is MMP-2, MMP-3, MMP-9, MMP-12 or MMP-13.

25. A method of treating an MMP-13-mediated disease or disorder in a mammal, said method comprising administering to said mammal a therapeutically effective amount of a compound as claimed in claim 1.

26. A method of inhibiting the production of MMP in a mammal, which comprises administering to said mammal an effective amount of a compound as claimed in claim 1.

27. The method of claim 26, wherein the MMP is MMP-2, MMP-3, MMP-9, MMP-12 or MMP-13.

28. A method of inhibiting the production of MMP-13 in a mammal, which comprises administering to said mammal an effective amount of a compound as claimed in claim 1.

29. A compound as claimed in claim 1 for use as a medicament in the treatment of MMP mediated conditions.

30. The compound of claim 29, wherein the MMP is MMP-2, MMP-3, MMP-9, MMP-12 or MMP-13.

31. A compound as claimed in claim 1 for use as a medicament in the treatment of MMP-13-mediated conditions.

32. The use of a compound as claimed in claim 1 in the manufacture of a medicament for the treatment of MMP mediated conditions.

33. The use of claim 32, wherein the MMP is MMP-2, MMP-3, MMP-9, MMP-12 or MMP-13.

34. The use of a compound as claimed in claim 1 in the manufacture of a medicament for the treatment of MMP-13-mediated conditions.

35. A method of preparing a compound of claim 1, comprising:
(a) coupling a compound having the Formula II: 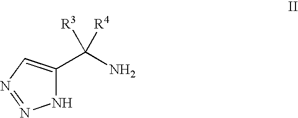
wherein R³ and R⁴ are as defined herein, with a compound having the Formula III: 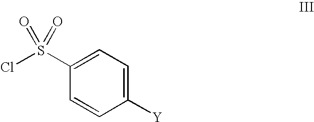
wherein Y is as defined herein, in the presence of a base; or
(b) reacting a compound having the Formula IV: 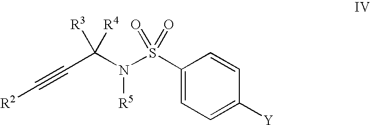
wherein R³, R⁴, R⁵, and Y are as defined herein and R² is not Br, with azidotrimethylsilane in the presence of a catalytic amount of CuI; or
(c) for compounds of Formula I wherein R² is Br and at least one of R³ and R⁴ is H, reacting a compound of Formula I wherein R² is H with bromine; and
removing any protecting group or groups and, if desired, forming a salt.

36. A pharmaceutical composition comprising a compound as claimed in claim

37. A method of treating an MMP-mediated disease or disorder in a mammal, said method comprising administering to said mammal a therapeutically effective amount of a compound as claimed in claim 21.

38. The method of claim 37, wherein the MMP is MMP-2, MMP-3, MMP-9, MMP-12 or MMP-13.

39. A method of treating an MMP-13-mediated disease or disorder in a mammal, said method comprising administering to said mammal a therapeutically effective amount of a compound as claimed in claim 21.

40. A method of inhibiting the production of MMP in a mammal, which comprises administering to said mammal an effective amount of a compound as claimed in claim 21.

41. The method of claim 40, wherein the MMP is MMP-2, MMP-3, MMP-9, MMP-12 or MMP-13.

42. A method of inhibiting the production of MMP-13 in a mammal, which comprises administering to said mammal an effective amount of a compound as claimed in claim

43. A compound as claimed in claim 21 for use as a medicament in the treatment of MMP mediated conditions.

44. The compound of claim 43, wherein the MMP is MMP-2, MMP-3, MMP-9, MMP-12 or MMP-13.

45. A compound as claimed in claim 21 for use as a medicament in the treatment of MMP-13-mediated conditions.

46. The use of a compound as claimed in claim 21 in the manufacture of a medicament for the treatment of MMP mediated conditions.

47. The use of claim 46, wherein the MMP is MMP-2, MMP-3, MMP-9, MMP-12 or MMP-13.

48. The use of a compound as claimed in claim 21 in the manufacture of a medicament for the treatment of MMP-13-mediated conditions.
